(19) United States
(12) Patent Application Publication
(10) Pub. No.: US 2020/0355703 A1
(43) Pub. Date: Nov. 12, 2020

(54) DETECTION OF INSULIN IN SALIVA AND METHODS OF USE THEREOF

(71) Applicant: 1083120 BC Ltd., Kelowna (CA)

(72) Inventor: Anne Marie Kirby, Kelowna (CA)

(73) Assignee: 1083120 BC Ltd., Kelowna, BC (CA)

(21) Appl. No.: 16/640,378

(22) PCT Filed: Aug. 20, 2018
(86) PCT No.: PCT/CA2018/051005

§ 371 Date: Feb. 20, 2020

Related U.S. Application Data
(60) Provisional application No. 62/548,643, filed on Aug. 22, 2017.

(51) Int. Cl. G01N 33/74

(52) U.S. Cl. CPC G01N 33/74; G01N 2800/042; G01N 2333/62

(57) ABSTRACT

A detection of insulin in saliva and methods of use thereof, may include methods for detecting or predicting a postprandial blood or plasma insulin response to a foodstuff, said method comprising: obtaining a first saliva sample from the subject; obtaining a second saliva sample from the subject following ingestion of a foodstuff; measuring a first level of insulin in the first saliva sample; measuring a second level of insulin in the second saliva sample; and characterizing the subject's blood or plasma insulin level based on a difference between the first level of insulin and the second level of insulin, to thereby detect or predict a postprandial blood or plasma insulin response to the foodstuff; the detection of insulin in saliva and methods of use thereof may also include methods for determining metabolic status, and methods for diagnosing and/or monitoring hyperinsulinemia in a subject.

### CROSS-REFERENCE TO RELATED APPLICATIONS

This application claims the benefit of and priority from U.S. Provisional Patent Application No. 62/548,643, filed Aug. 22, 2017, which is incorporated herein by reference.

### FIELD

This disclosure generally relates to the detection of insulin in saliva and methods of use thereof, including detecting or predicting a postprandial blood or plasma insulin response to a foodstuff, determining metabolic status, or diagnosing and/or monitoring hyperinsulinemia in a subject.

### BACKGROUND

Obesity has become a worldwide problem with recent estimates suggesting that roughly 500 million adults are obese, i.e. having a body mass index (BMI) of 30 or higher. Although the occurrence and severity of obesity may be affected by genetic, behavioral and hormonal influences on body weight, the primary causes of obesity are a regular on-going intake of more calories than are burned through exercise and normal daily activities combined with a sedentary lifestyle, resulting in the storage of excess calories as fat. The problems with obesity are exacerbated by high-calorie diets, and the consumption of fast food and high-calorie beverages.

Most obese individuals sporadically undertake various types of dieting programs in attempts to lose weight and improve their health. Most dieting programs are focused on reducing the intake of calories which they consume and/or on increasing the number of calories which they burn. However, estimates indicate that approximately 90 percent of people who follow a diet plan and consequently lose weight, subsequently put back any weight that was lost when they stop following the diet plan.

Some of the problems with dieting programs include reductions or restrictions in permitted quantities or volumes of foods, significant increases in high-fiber and/or roughage food volumes, common occurrences of perceived lack of sufficient nutrition evidenced by increasing occurrence of hunger pangs, and imbalances between the food intake events with individuals' physiological conditions and activities.

Compelling data show that elevated insulin levels are associated with the development of pathological conditions, including obesity and type 2 diabetes (Corkey, 2012a). For the past few decades, hyperinsulineamia has been mainly considered as a consequence of obesity but this concept has recently been revisited (Templeman, 2017). A revised model of obesity and type 2 diabetes suggests a more central and causal role of hyperinsulinemia, which is thought to precede and drive metabolic abnormalities (Mehran, 2012; Corkey, 2012b). Insulin hypersecretion and insulin resistance are detectable up to several years prior to abnormalities in glucose tolerance [Warram, 1900; Lillioja, 1993; Kashyap, 2004; Gulli, 1992; Pratipanawatz, 2001). Over time, persistent elevated insulin secretion can no longer be maintained by pancreatic beta-cells, leading to chronic hyperglycemia and the associated diagnoses of prediabetes or type 2 diabetes (Kahn, 2006), which significantly increases the risk for cardiovascular disease and mortality (Centers for Disease Control and Prevention, 2011). Thus, elevations in basal and stimulated insulin levels may constitute an important early marker of metabolic dysfunction that could be monitored in both apparently healthy and at-risk individuals.

The theoretical and practical importance of postprandial insulin levels have previously been emphasized by proposing the use of a food insulin index that ranks foods based on their ability to elevate postprandial insulin (Holt, 1997; Bao, 2009). Although potentially useful, a general food insulin index based on the postprandial blood insulin responses to isolated foods in healthy volunteers (Holt, 1997) would not take into account the inter-individual variability in insulin secretion and/or responses to mixed meals, nor would it be able to account for expected differences in postprandial insulin levels between individuals with different levels of insulin resistance. From a clinical and monitoring perspective, measuring postprandial insulin levels would be highly valuable but presents several challenges, including the requirement for repeated blood sampling. Developing non-invasive, user-friendly alternatives to detect, determine or predict postprandial blood or plasma insulin levels in humans therefore holds potential value.

### SUMMARY

The embodiments of the present disclosure generally relate to the detection of insulin in saliva and methods of use thereof, including detecting or predicting a postprandial blood or plasma insulin response to a foodstuff; determining metabolic status, such as determining whether an individual's physiological condition is in a calorie-assimilation (fat-storing) mode or a calorie-burning (fat-burning) mode; or diagnosing and/or monitoring hyperinsulinemia in a subject.

One embodiment relates to a method for detecting or predicting a postprandial blood or plasma insulin response to a foodstuff in a subject, said method comprising: measuring a level of insulin in saliva from a subject following ingestion of a foodstuff; and characterizing the subject's blood or plasma insulin level based upon the level of insulin in saliva, to thereby detect or predict a postprandial blood or plasma insulin response to the foodstuff.

Another embodiment relates to a method for determining whether a subject is in a fat-storage mode or a fat-burning mode, said method comprising: measuring a level of insulin in saliva from a subject; and comparing the level of insulin in saliva to a predetermined value to determine if the subject is in a fat-storage mode or a fat-burning mode, wherein a level of insulin in saliva above the predetermined value is indicative of a fat-storage mode and a level of insulin in saliva below the predetermined value is indicative of a fat-burning mode.

Another embodiment relates to a method for diagnosing and/or monitoring hyperinsulinemia in a subject, said method comprising measuring a level of insulin in saliva from a subject following ingestion of a high carbohydrate meal and/or a low-carbohydrate meal.

### BRIEF DESCRIPTION OF THE DRAWINGS

Further advantages, permutations and combinations of the invention will now appear from the above and from the following detailed description of the various particular embodiments of the invention taken together with the accompanying drawings, each of which are intended to be non-limiting, in which:

FIG. 1 depicts charts showing plasma and saliva insulin responses over 120 minutes to low-carbohydrate (LC) and high-carbohydrate (HC) breakfast meals in lean (N=8) and obese (N=7) human participants. FIG. 1A shows plasma insulin response in lean individuals, FIG. 1B shows plasma insulin response in obese individuals, FIG. 1C shows saliva insulin response in lean individuals, and FIG. 1D shows saliva insulin response in obese individuals;

FIG. 2 depicts charts showing plasma and saliva insulin levels of all subject groups. FIG. 2A shows changes in plasma insulin levels over a 2-h period while FIG. 2B shows changes in saliva insulin levels over a 2-h period in: (i) a lean group after a high-carbohydrate meal, (ii) a lean group after a low-carbohydrate meal, (iii) an obese group after a high-carbohydrate meal, and (iv) an obese group after a low-carbohydrate meal;

FIG. 3 depicts charts showing plasma and saliva insulin of low-carbohydrate subject groups. FIG. 3A compares plasma insulin levels over a 2-h period in the lean group and the obese group after a low-carbohydrate meal, while FIG. 3B shows changes in saliva insulin levels over a 2-h period in the lean group and the obese group after a low-carbohydrate meal;

FIG. 4 depicts charts showing plasma and saliva insulin of high-carbohydrate subject groups. FIG. 4A compares plasma insulin levels over a 2-h period in the lean group and the obese group after a high-carbohydrate meal, while FIG. 4B shows changes in saliva insulin levels over a 2-h period in the lean group and the obese group after a high-carbohydrate meal;

FIG. 5 depicts charts showing plasma and saliva insulin of lean subject groups. FIG. 5A compares plasma insulin levels over a 2-h period in the first lean group after a low-carbohydrate meal and the second lean group after a high-carbohydrate meal, while FIG. 5B shows changes in saliva insulin levels over a 2-h period in the first lean group after a low-carbohydrate meal and the second lean group after a high-carbohydrate meal. * in FIG. 5A denotes that all time points were different from time 0 min, only in the high-carbohydrate condition. * in FIG. 5B denotes that times 60, 90 and 120 min were different from time 0 min, only in the high-carbohydrate condition;

FIG. 6 depicts charts showing plasma and saliva insulin of obese subject groups. FIG. 6A compares plasma insulin levels over a 2-h period in the first obese group after a low-carbohydrate meal and the second obese group after a high-carbohydrate meal, while FIG. 6B shows changes in saliva insulin levels over a 2-h period in the first obese group after a low-carbohydrate meal and the second obese group after a high-carbohydrate meal. * in FIG. 6A denotes that all time points were different from time 0 min, only in the high-carbohydrate condition. * in FIG. 6B denotes that times 60 and 90 min were different from time 0 min, only in the high-carbohydrate condition;

FIG. 7 is a chart comparing plasma glucose levels over a 2-hr period in (i) the lean group after a high-carbohydrate meal, (ii) the lean group after a low-carbohydrate meal, (iii) the obese group after a high-carbohydrate meal, and (iv) the obese group after a low-carbohydrate meal;

FIG. 8 depicts charts showing plasma glucose levels of subject groups. FIG. 8A compares plasma glucose levels over a 2-hr period of the lean group with the obese group after a low-carbohydrate meal, while FIG. 8B compares plasma glucose levels over a 2-hr period of the lean group with the obese group after a high-carbohydrate meal;

FIG. 9 is a chart comparing plasma glucose levels over a 2-hr period of the lean group after a low-carbohydrate meal with the lean group after a high-carbohydrate meal;

FIG. 10 is a chart comparing plasma glucose levels over a 2-hr period of the obese group after a low-carbohydrate meal with the obese group after a high-carbohydrate meal;

FIG. 11 depicts charts showing plasma and saliva insulin area under the curve (AUC) following low-carbohydrate (LC) and high-carbohydrate (HC) breakfast meals in lean (N=8) and obese (N=7) human participants. FIG. 11A shows plasma insulin total AUC, FIG. 11B shows plasma insulin incremental AUC (iAUC), FIG. 11C shows saliva insulin total AUC, and FIG. 11D shows saliva insulin iAUC in lean and obese individuals. # denotes a main effect of group (obese higher than lean, p<0.05). * denotes a main effect of meal (HC higher than LC);

FIG. 12 depicts charts showing plasma glucose responses over 120 minutes to low-carbohydrate (LC) and high-carbohydrate (HC) breakfast meals in lean (N=8) and obese (N=7) human participants. FIG. 12A shows plasma glucose response in lean individuals and FIG. 12B shows plasma glucose response in obese individuals;

FIG. 13 depicts charts showing relationships between plasma and saliva insulin in combined lean and obese participants (N=15). FIG. 13A shows fasting insulin, FIG. 13B shows total insulin area under the curve (AUC) following the high-carbohydrate (HC) meal, and FIG. 13C shows incremental AUC (iAUC) following the HC meal. Significant correlations were detected between plasma and saliva insulin;

FIG. 14 shows the average plasma versus saliva insulin of 8 healthy normal weight male subjects of the high-carbohydrate meal condition (HC) whereby the saliva insulin values are neither multiplied nor shifted back;

FIG. 15 shows the average plasma versus saliva insulin of 8 healthy normal weight male subjects of the high-carbohydrate meal whereby the saliva insulin values are multiplied by 4 (×4) and shifted back by 30 minutes;

FIG. 16 shows the average plasma versus saliva insulin of all 8 healthy normal weight male subjects of the high-carbohydrate meal and active condition (ACT) whereby the saliva insulin values are neither multiplied nor shifted back;

FIG. 17 shows the average plasma versus saliva insulin of 8 healthy normal weight male subjects of the high-carbohydrate meal and active condition (ACT) whereby the saliva insulin values are multiplied by 4 (×4) and shifted back by 30 minutes;

FIG. 18 shows the average plasma versus saliva insulin of 8 healthy normal weight male subjects of the low-carbohydrate meal condition (LC) whereby the saliva insulin values are neither multiplied nor shifted back;

FIG. 19 shows the average plasma versus saliva insulin of 8 healthy normal weight male subjects of the low-carbohydrate meal condition (LC) whereby the saliva insulin values are multiplied by 2 (×2) and shifted back by 30 minutes;

FIG. 20 shows the saliva insulin profile for subject 1 with elevated waist circumference (EWC) for each of the high-carbohydrate sedentary (HC), high-carbohydrate active (ACT) and low-carbohydrate (LC) conditions;

FIG. 21 shows the saliva insulin profile for subject 2 with elevated waist circumference (EWC) for each of the high-carbohydrate sedentary (HC), high-carbohydrate active (ACT) and low-carbohydrate (LC) conditions;

FIG. 22 shows the saliva insulin profile for subject 3 with elevated waist circumference (EWC) for each of the high-carbohydrate sedentary (HC), high-carbohydrate active (ACT) and low-carbohydrate (LC) conditions;

FIG. 23 shows the saliva insulin profile for subject 4 with elevated waist circumference (EWC) for each of the high-carbohydrate sedentary (HC), high-carbohydrate active (ACT) and low-carbohydrate (LC) conditions;

FIG. 24 shows the plasma insulin profile for subject 1 with elevated waist circumference (EWC) for each of the high-carbohydrate sedentary (HC), high-carbohydrate active (ACT) and low-carbohydrate (LC) conditions;

FIG. 25 shows the plasma insulin profile for subject 3 with elevated waist circumference (EWC) for each of the high-carbohydrate sedentary (HC), high-carbohydrate active (ACT) and low-carbohydrate (LC) conditions;

FIG. 26 shows the plasma versus saliva insulin profile of subject 1 with elevated waist circumference (EWC) for the high-carbohydrate sedentary (HC) condition;

FIG. 27 shows the plasma versus saliva insulin profile of subject 1 with elevated waist circumference (EWC) for the high-carbohydrate active (ACT) condition;

FIG. 28 shows the plasma versus saliva insulin profile of subject 1 with elevated waist circumference (EWC) for the low-carbohydrate (LC) condition;

FIG. 29 shows the plasma versus saliva insulin profile of subject 3 with elevated waist circumference (EWC) for the high-carbohydrate sedentary (HC) condition;

FIG. 30 shows the plasma versus saliva insulin profile of subject 3 with elevated waist circumference (EWC) for the high-carbohydrate active (ACT) condition;

FIG. 31 shows the plasma versus saliva insulin profile of subject 3 with elevated waist circumference (EWC) for the low-carbohydrate (LC) condition;

FIG. 32 shows the plasma versus saliva insulin profile of subject 1 with elevated waist circumference (EWC) for the high-carbohydrate sedentary (HC) condition whereby the saliva insulin values are shifted back by 30 minutes;

FIG. 33 shows the plasma versus saliva insulin profile of subject 1 with elevated waist circumference (EWC) for the high-carbohydrate active (ACT) condition whereby the saliva insulin values are shifted back by 30 minutes;

FIG. 34 shows the plasma versus saliva insulin profile of subject 3 with elevated waist circumference (EWC) for the high-carbohydrate sedentary (HC) condition whereby the saliva insulin values are shifted back by 60 minutes;

FIG. 35 shows the plasma versus saliva insulin profile of subject 1 with elevated waist circumference (EWC) for the high-carbohydrate active (ACT) condition whereby the saliva insulin values are shifted back by 30 minutes;

FIG. 36 shows the average saliva insulin profile of healthy normal weight male subjects versus subjects with elevated waist circumference during high-carbohydrate sedentary (HC) condition;

FIG. 37 shows the average saliva insulin profile of healthy normal weight male subjects versus subjects with elevated waist circumference during high-carbohydrate active (ACT) condition;

FIG. 38 shows the average saliva insulin profile of healthy normal weight male subjects versus subjects with elevated waist circumference during low-carbohydrate (LC) condition;

FIG. 39 shows the saliva insulin profile of subjects with elevated waist circumference during high-carbohydrate sedentary (HC) condition;

FIG. 40 shows the saliva insulin profile of subjects with elevated waist circumference during high-carbohydrate active (ACT) condition;

FIG. 41 shows the saliva insulin profile of subjects with elevated waist circumference during low-carbohydrate (LC) condition;

FIG. 42 shows area under the curve (AUC) plots for plasma (Y-axis) versus saliva (X-axis) insulin profiles for the total 9-hour day for the healthy normal weight male subjects under each of the conditions (HC, ACT and LC);

FIG. 43 shows AUC plots for plasma (Y-axis) versus saliva (X-axis) insulin profiles for 3-hours following breakfast for the healthy normal weight male subjects under each of the conditions (HC, ACT and LC);

FIG. 44 shows incremental area under the curve (iAUC) plots for plasma (Y-axis) versus saliva (X-axis) insulin profiles for 3-hours following breakfast for the healthy normal weight male subjects under each of the conditions (HC, ACT and LC);

FIG. 45 shows AUC plots for plasma (Y-axis) versus saliva (X-axis) insulin profiles for 3-hours following lunch for the healthy normal weight male subjects under each of the conditions (HC, ACT and LC);

FIG. 46 shows incremental area under the curve (iAUC) plots for plasma (Y-axis) versus saliva (X-axis) insulin profiles for 3-hours following breakfast for the healthy normal weight male subjects under each of the conditions (HC, ACT and LC);

FIG. 47 shows AUC plots for plasma (Y-axis) versus saliva (X-axis) insulin profiles for 3-hours following dinner for the healthy normal weight male subjects under each of the conditions (HC, ACT and LC);

FIG. 48 shows incremental area under the curve (iAUC) plots for plasma (Y-axis) versus saliva (X-axis) insulin profiles for 3-hours following breakfast for the healthy normal weight male subjects under each of the conditions (HC, ACT and LC);

FIG. 49 shows plots depicting the correlation of fasting plasma (Y-axis) versus fasting saliva (X-axis) on days 1, 2 and 3 for the healthy normal weight male subjects;

FIG. 50 shows a plot depicting the correlation of fasting plasma (Y-axis) versus saliva (X-axis) for all of the participants in the study of Example 1 and the healthy normal weight male subjects of Example 2 (FIG. 53A) and a plot depicting AUC for plasma (Y-axis) versus saliva (X-axis) insulin profiles in HC condition after breakfast meal for participants in the study of Example 1 and the healthy normal weight male subjects of Example 2 (FIG. 53B); and

FIG. 51 shows AUC plots for plasma (Y-axis) versus saliva (X-axis) insulin profiles for Total 9-hours for males with healthy normal weight and subjects with elevated waist circumference for each of the high-carbohydrate sedentary (HC), high-carbohydrate active (ACT) and low-carbohydrate (LC) conditions.

### DETAILED DESCRIPTION

Most diet plans provide spaced-apart food consumption events, most commonly three times per day, of foodstuffs comprising a balance of carbohydrates, proteins and fats. Digestion typically starts in the mouth wherein starches begin to be broken down into monosaccharides and disaccharides by enzymes present in the saliva. Digestion of some fats, which are mostly triglycerides, also begins in the mouth wherein some short-chain lipids are broken down into diglycerides. Proteins are broken down into polypeptides in the stomach, which are then further broken into dipeptides and amino acids in the duodenum. The majority of the carbohydrates and fats are broken down in the duodenum into small molecules such as monosaccharides, disaccharides, monoglycerides, and diglycerides. These small molecules are further processed before absorption into the body's vascular system from the small intestines.

Absorption of glucose into the bloodstream as a consequence of the digestive process, begins to rise about 15 min after food intake occurs and typically peaks about one hour after a balanced food consumption event. The increase in blood sugar (i.e. glucose) stimulates release of the hormone insulin from the pancreas into the blood stream. Insulin in the bloodstream causes the glucose to be taken up into body tissues, for example into the liver and skeletal muscles, thereby causing the blood sugar level to return to its normal resting levels within two to four hours after the food consumption event. Insulin, however, has other effects on the body's metabolism. Insulin also regulates fat metabolism by inhibiting the breakdown of triglycerides within fat cells (i.e. adipose tissues) and by stimulating absorption of some of the fatty acids and glucose from the blood stream to additional fat which is stored in the liver. Therefore, the impacts of high insulin levels on fat metabolism are to inhibit fat breakdown (i.e. inhibit “lipolysis”) and to promote fat synthesis (i.e. increase “lipogenesis”). Such physiological processes stimulated when insulin levels are high, are commonly referred to as a “fat-storage” mode. Maintaining low insulin levels is believed to encourage a state of “fat-burning” and prevents the accumulation of excess body fat. The pancreas produces a second hormone, glucagon which generally functions to oppose the effects of insulin. As the levels of blood sugar fall to the normal resting level or lower, the pancreas is stimulated to produce and release glucagon into the blood stream. As glucagon levels increase in the bloodstream, the insulin levels drop. Increasing glucagon levels have two primary effects: first, glycogen stored in the liver is converted into glucose that is released into the bloodstream, and second, the synthesis of glucose from other sources is stimulated, for example from amino acids and fat metabolites. The effects of glucagon on increasing blood sugar levels concurrent with increased lipolysis, are commonly referred to as a “fat-burning” mode.

Accordingly, it would seem that detecting and quantifying increased or decreased levels of insulin in the bloodstream would be a useful indication of whether an individual's metabolism was in the “fat-storage” mode or a “fat-burning” mode. Having this information readily available to an individual (e.g. who is dieting) would allow them to make decisions regarding the timing of their food consumption events and also, the types of foods they should be considering for intake. For example, unprocessed foods with a low glycemic index or foods with a low carbohydrate content will tend to raise blood sugar marginally, and consequently will result in a marginal increase in the bloodstream levels of insulin. On the other hand, processed foods and foods with a high glycemic index will cause substantial increases in blood sugar that will be accompanied by significant increases in the bloodstream levels of insulin. Although these general rules can help to predict whether a specific food will elicit a low or a high insulin response, the degree of hyperinsulinemia following food consumption is highly variable between individuals and also, is dependent on their baseline levels of insulin sensitivity and pancreatic beta-cell function. Insulin responses are therefore highly individualized and accurate determination of a person's physiological state generally requires actually measuring their blood or plasma insulin levels.

A problem is that present reliable methods for detecting and quantifying insulin are based on performing ELISA assays or radioimmunoassays on blood samples. Such assays typically take about 4-5 hrs to perform after a blood sample is drawn, are expensive, and suffer from patient compliance issues with respect to blood sampling. While it is known that insulin is present in and can be detected in saliva, it is not known if the insulin levels in the saliva are correlated with levels of insulin in the blood after eating different types of foods. In particular, it was not known whether changes in saliva insulin are sensitive enough to delineate between meals with different insulin responses or whether subtle differences in postprandial insulin levels, which might reflect systemic insulin resistance or increased metabolic risk (e.g. hyperinsulinemia), are detectable by saliva insulin measures.

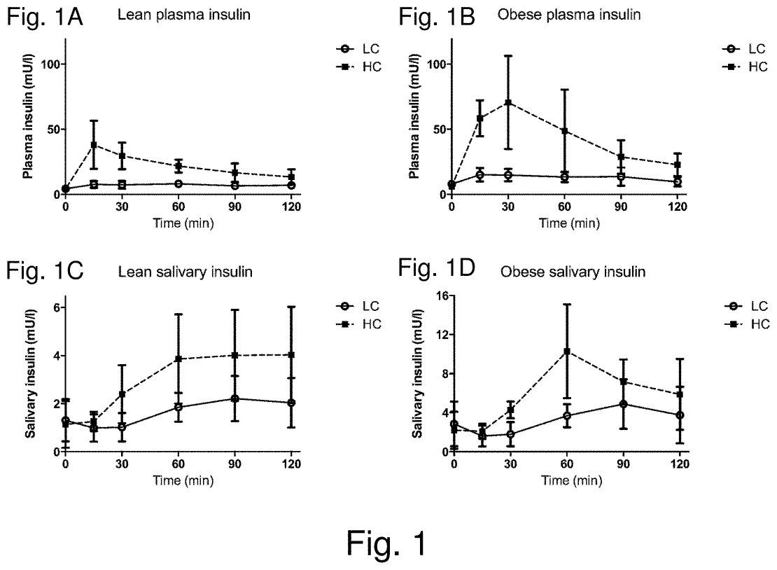

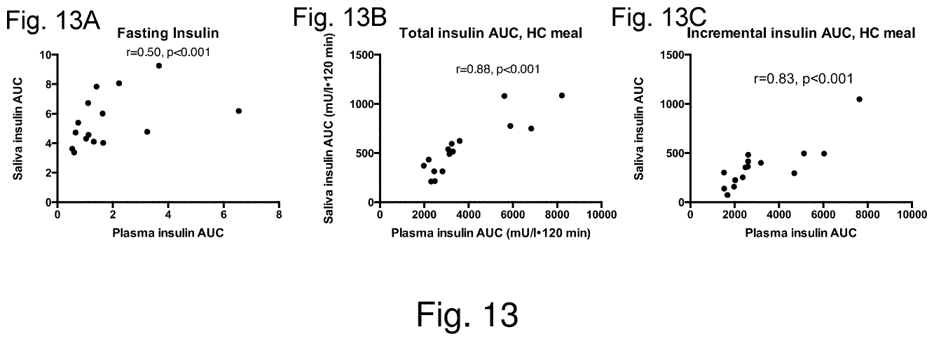

As disclosed herein, salivary insulin was found to be capable of delineating between high and low insulin levels following the ingestion of a high- and low-carbohydrate mixed meal (see e.g. FIG. 1). The examples herein consistently demonstrated that differences in saliva insulin responses to meals were evident, e.g. the high-carbohydrate meal led to larger postprandial insulin responses compared to the low-carbohydrate meal and the obese subjects had higher insulin responses than the lean subjects. Moreover, fasting insulin differences between lean and obese subject groups were detectable in both plasma and salivary samples, with higher levels of insulin observed in the obese group as compared to the lean group (see e.g. Table 2). As shown in Table 2, fasting salivary insulin concentration was about 30% of the plasma concentration. Nonetheless, fasting saliva and plasma insulin were positively correlated (FIG. 13; r=0.60, p=0.017) indicating that individuals with the higher basal plasma insulin levels also had higher salivary insulin levels. Indeed, significant correlations between saliva and plasma insulin were found for fasting insulin and the insulin area under the curve (AUC) after both the high-carbohydrate and low-carbohydrate meal.

In addition, significant MEAL×GROUP interactions were observed herein. Carbohydrates are known as the most potent stimulator of insulin secretion (Pallotta, 1968) with increased secretion observed when combined to insulinotropic amino acids (Van Loon, 2000). As shown herein, the high-carbohydrate meal increased the total and incremental insulin AUC over the 2-hour postprandial period as compared to the low-carbohydrate meal in both groups with a greater increase in the obese group as compared to the lean group. Importantly, these differences were observed in both fluid samples (plasma and saliva) demonstrating that differences in blood insulin levels following the consumption of distinct meals types (e.g. high versus low carbohydrate) can detected, determined and predicted based on saliva insulin levels. Thus, the present invention shows that differences in salivary insulin can be tracked between meals with different insulinogenic effects and between individuals with significantly different postprandial insulin responses.

Along those lines, significant correlations were observed between plasma and salivary insulin total AUC (LC: r=0.821 and HC r=0.882) and total iAUC (HC: r=0.731). The absence of significant correlation in iAUC after the LC meal (r=0.126) can be explained by the small insulin excursion following the LC meal, which meant that variability in incremental insulin response between participants was close to zero. Generally speaking, it was found that the peak insulin response in saliva was delayed by 30-45 minutes relative to blood.

The absence of glucose differences between groups, combined with the higher insulin levels in the obese subjects as compared to the lean subjects, indicates that the obese subjects were able to successfully compensate for a higher degree of insulin resistance. Moreover, these results indicate that some metabolic impairments can be present, and detected with both plasma and saliva insulin measurements (e.g. hyperinsulinemia), in overweight to obese but otherwise healthy, young and normoglycemic participants.

It was further found that saliva insulin can be used to reflect plasma insulin responses to separate meals throughout the day, and that the validity of the insulin responses were not altered by performing exercise (Example 2). Overall, saliva insulin tends to be lower than the value measured in plasma insulin, and excursions after meals for saliva insulin levels tend to lag about 30 minutes behind changes in plasma insulin levels. Also, baseline and post-meal saliva insulin was higher in subjects with elevated waist circumference as compared to healthy normal weight male subjects, but returned to baseline levels almost 3 hours after each meal.

The peak saliva insulin response after a high-carbohydrate meal (e.g. at breakfast, lunch or dinner) occurs at about 60-90 minutes post-meal. Levels above basal would be indicative of a food-storage mode and subjects on a diet program would benefit from avoiding ingestion of foodstuffs at that time. After breakfast, lunch and dinner meals the saliva insulin levels appear to return to basal at approximately 180 minutes post-meal. Since saliva insulin levels lag plasma levels by about 30 minutes, this indicates that such subjects may commence a fat-burning mode after 150 minutes post-meal, and subjects on a diet should wait at least as long as this period before ingestion of further foodstuffs. With repeated monitoring of saliva insulin levels, real-time information on a subject's physiological condition can be more accurately followed, e.g. to determine if a particular subject is in a fat-storage or fat-burning mode at any point in time.

Moreover, elevated saliva insulin levels that are outside the norm for a subject of a certain physiological condition (e.g. gender, age, weight, body mass index, waist-to-hip ratio, systolic blood pressure, diastolic blood pressure, metabolism, race, and exercise profile) after a high-carbohydrate and/or low-carbohydrate meal may be reflective of an underlying metabolic disorder, such as for example hyperinsulinemia (prediabetes).

In embodiments therefore, the present invention relates to the detection of insulin in saliva and methods of use thereof, including detecting or predicting a postprandial blood or plasma insulin response to a foodstuff; determining metabolic status, such as determining whether an individual's physiological condition is in a calorie-assimilation (fat-storing) mode or a calorie-burning (fat-burning) mode; or diagnosing and/or monitoring hyperinsulinemia in a subject.

One embodiment relates to a method for detecting or predicting a postprandial blood or plasma insulin response to a foodstuff in a subject, said method comprising: measuring a level of insulin in saliva from a subject following ingestion of a foodstuff; and characterizing the subject's blood or plasma insulin level based upon the level of insulin in saliva, to thereby detect or predict a postprandial blood or plasma insulin response to the foodstuff.

As used herein, the term “detecting” is used in the context of determining or ascertaining a postprandial blood or plasma insulin response at a particular point in time or over a particular period of time after ingestion of a foodstuff by the subject. For example, in an embodiment, by “detecting” it is meant that the disclosed methods determine or ascertain the postprandial insulin response to the particular foodstuff that was ingested by the subject. “Detecting” is used interchangeably herein with “detect” or “detection” and means to identify, determine or ascertain a postprandial blood or plasma insulin level (e.g. quantity), profile or relationship to the foodstuff. In a particular embodiment, detecting means to determine the exact or approximate quantity of postprandial insulin in the blood or plasma based on the saliva insulin level, in accordance with the disclosed methods.

As used herein, the term “predicting” is distinguished from detecting in that by “predicting” it is meant that information or data obtained from the disclosed methods is used to establish the likelihood of future events. For example, in an embodiment “predicting” refers to the ability to identify, determine or ascertain a subsequent postprandial blood or plasma insulin response to a foodstuff by a particular subject. In another embodiment, “predicting” refers to the ability to identify, determine or ascertain a postprandial blood or plasma insulin response to a foodstuff by a different subject, such as a subject having a similar physiological condition or profile to a reference subject or reference data set. In another embodiment, “predicting” refers to the ability to identify, determine or ascertain a postprandial blood or plasma insulin response to a similar foodstuff as tested, whether by the same or different subject. “Predicting” is used interchangeably herein with “predict” and means to identify, determine or ascertain a future postprandial blood or plasma insulin level (e.g. quantity), profile or relationship to the same or similar foodstuff in the same or different subject.

By “similar foodstuff”, it is meant a foodstuff that minimally has a similar carbohydrate content as the tested foodstuff. In an embodiment, similar carbohydrate content means that the foodstuff has a carbohydrate content by weight that is within about 10% of the tested foodstuff. More particularly, the carbohydrate content is within about 9%, about 8%, about 7%, about 6%, about 5%, about 4%, about 3%, about 2% or about 1% of the tested foodstuff. In an embodiment, similar carbohydrate content means that the foodstuff contains a similar composition of carbohydrates as the tested foodstuff. For example, the similar foodstuff may contain a similar quantity of fast-acting/rapid-absorption sugars (e.g. glucose), slow-acting/slow-absorption sugars (e.g. starch) and/or sugars having a similar glycemic index. By “similar quantity” it means that the quantity is within about 10%, about 9%, about 8%, about 7%, about 6%, about 5%, about 4%, about 3%, about 2% or about 1% by weight.

In addition to a similar carbohydrate content, the similar foodstuff may also have one or more of a similar quantity of protein, fiber, calories and fat. Again, by “similar quantity” it means that the quantity is within about 9%, about 8%, about 7%, about 6%, about 5%, about 4%, about 3%, about 2% or about 1% by weight.

By “similar physiological condition or profile”, it is meant that subjects have in common one or more of the following characteristics: gender, age (within 5 years), weight (within 25 kg), body mass index (within 5 kg/m²), waist-to-hip ratio (within 0.1 cm), systolic blood pressure (within 10 mmHg), diastolic blood pressure (within 10 mmHg), metabolism, race, and exercise (within 5 hours per week). In an embodiment, a subject having a similar physiological condition may share only one of these characteristics. In an embodiment, a subject having a similar physiological profile may share two or more of these characteristics. In an embodiment, a subject having a similar physiological profile may share three, four, five, six, seven, eight, nine or all ten of these characteristics.

Embodiments of the methods disclosed herein are for detecting or predicting a postprandial blood or plasma insulin response to a foodstuff in a subject. The terms “postprandial”, “blood” and “plasma” have their ordinary meaning in the art. Namely, “postprandial” refers to the period during or after ingestion of a foodstuff (e.g. a meal). In an embodiment, postprandial is the period within about 30 minutes, about 60 minutes, about 90 minutes, about 120 minutes, about 150 minutes or about 180 minutes after consumption of a foodstuff, or any shorter or longer time period prior to the consumption of a subsequent foodstuff. The term “blood” refers to the bodily fluid responsible for transporting nutrients and oxygen to the cells of the body and metabolic waste away from these same cells, such as within the vascular system. “Plasma” refers to the yellow liquid component of blood and constitutes approximately 55% of total blood volume.

As used herein, the term “foodstuff” refers to any substance that is suitable for consumption as a food. The term includes solids, liquids and mixtures thereof. The term “foodstuff” is used interchangeably herein with the term “meal”, which also includes both food and drink. The term “subject” refers to any animal In an embodiment, the subject is a human.

The methods disclosed herein for detecting or predicting a postprandial blood or plasma insulin response to a foodstuff in a subject comprise measuring a level of insulin in saliva. This step of measuring insulin in saliva may be performed by any means available in the art and may include in vivo, in vitro and immunological methods. In the in vitro methods for example, an isolated tissue may by incubated with the saliva and the effect of insulin in the saliva measured by one or more of the metabolic processes of the tissue in comparison to a standard. In the immunological methods for example, insulin in the saliva is determined or quantified by its binding to an insulin antibody.

In an embodiment of the methods disclosed herein, the insulin level in saliva may be determined by enzyme-linked immunosorbent assay (ELISA), such as for example and without limitation a human insulin ELISA kit that is commercially available (e.g. from Invitrogen, Abeam, Mercodia, Crystal Chem and Millipore). In an embodiment, saliva insulin levels may be determined using the Mercodia Ultrasensitive Human Insulin ELISA or the Crystal Chem Human Insulin ELISA. In a particular embodiment, the Mercodia Ultrasensitive Human Insulin ELISA may be used as detailed in the examples herein. It is within the ability of the skilled person to determine sample dilutions that are suitable for the respective ELISA assays. In an embodiment, the sample may be diluted 1:1, 1:2, 1:3, 1:4, 1:5, 1:10, 1:25, 1:50 or 1:100. In a particular embodiment, the sample may be diluted 1:2 or 1:4.

In another embodiment of the methods disclosed herein, the insulin level in saliva may be determined by use of a proximity litigation assay. For example, in an embodiment, the level of insulin in saliva may be determined by an Electrochemical Proximity Assay (ECPA), such as without limitation the EPCA developed by Auburn University (see e.g. U.S. Pat. No. 9,335,292; Hu, 2012; Hu, 2014) , modified for insulin detection. In another embodiment, the level of insulin in saliva may be determined by thermally resolved molecule assays, such as disclosed in WO 2016/073594, modified for insulin detection.

In another embodiment of the methods disclosed herein, the insulin level in saliva may be determined by use of the system or device described in U.S. Provisional Patent Application No. 62/548,643, filed Aug. 22, 2017, which is incorporated herein by reference.

Where it is desired to determine the level of blood or plasma insulin levels by actual detection of these levels in a subject, such as described below in the generation of a reference data set, methods of blood or plasma insulin detection are known and available in the art. As well, the techniques described above for determining the level of insulin in saliva may be used for the determination of blood or plasma insulin levels, adapted accordingly.

By “level of insulin in saliva”, it is generally meant to refer to the quantity of insulin in saliva, such as measured in picomole per litre (pmol/l), millimole per litre (mmol/l) or milliunits per litre (mU/l). In other embodiments, “level of insulin in saliva” may refer to the activity of insulin in a saliva sample. This measure of activity may then be used to determine the quantity based on comparison to a standard or reference sample.

In order to measure a level of insulin in saliva, the methods disclosed herein may further comprise a step of obtaining a saliva sample from the subject. The saliva sample may be of any quantity suitable or necessary to measure the level of insulin in saliva. In an embodiment, the saliva sample may be collected from a subject by passive drool for a period of about 5 seconds, about 10 seconds, about 15 seconds, about 20 seconds, about 25 seconds, about 30 seconds, about 35 seconds, about 40 seconds, about 45 seconds, about 50 seconds, about 55 seconds, about 60 seconds, or longer. In an embodiment, the saliva sample may be collected by touching a collection apparatus to a surface of the mouth, tongue or other surface of the buccal cavity.

In an embodiment, the step of measuring the level of insulin in saliva is performed on saliva taken from a subject at any time after ingestion of a foodstuff and prior to ingestion of a further foodstuff. In an embodiment, the step of measuring the level of insulin in saliva is performed on saliva taken from a subject at about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 165 minutes, about 170 minutes, about 175 minutes or about 180 minutes after ingestion of a foodstuff. In an embodiment, the step of measuring the level of insulin in saliva is performed on saliva taken from a subject at least 15 minutes, 30 minutes, 45 minutes, 60 minutes, 75 minutes, 90 minutes, 105 minutes, 120 minutes, 135 minutes, 150 minutes, 165 minutes or 180 minutes after ingestion of a foodstuff In a particular embodiment, the step of measuring the level of insulin in saliva is performed on saliva taken from a subject at least 30 minutes after ingestion of a foodstuff.

In an embodiment, the step of measuring the level of insulin in saliva from a subject may be repeated any number of times prior to the consumption of a subsequent foodstuff In an embodiment, the step of measuring the level of insulin in saliva from a subject is repeated 1, 2, 3, 4, 5, 6, 7, 8, 9, 10 or more times by measuring insulin in saliva taken from the subject at different times. In an embodiment, the step of measuring the level of insulin in saliva is repeated on saliva taken from the subject once every 5 minutes, 10 minutes, 15 minutes, 30 minutes, 45 minutes or 60 minutes. In a particular embodiment, the step of measuring the level of insulin in saliva is repeated on saliva taken from a subject at about 30 minutes post-meal and every 30 minutes thereafter until 3 hours post-meal or until the next meal is consumed.

The methods disclosed herein for detecting or predicting a postprandial blood or plasma insulin response to a foodstuff in a subject comprise characterizing the subject's blood or plasma insulin level based upon the level of insulin in saliva. The step of characterizing the subject's blood or plasma insulin level may be performed in a number of ways based on the findings of the present invention.

In an embodiment, the step of characterizing the subject's blood or plasma insulin level comprises comparing the level of insulin in saliva from the subject to a reference data set. The reference data set may be compiled based on data from an individual subject or based on data from a group of subjects that share a similar physiological condition or profile, as defined elsewhere herein.

In an embodiment, the reference data set may be compiled from comparisons of plasma and saliva insulin levels in the subject at baseline and in response to different types of foodstuffs (high-carbohydrate and low-carbohydrate meals) at one or more different times post-meal. This may be used to generate a unique insulin response profile for the individual which characterizes various features, such as and without limitation: (i) saliva insulin lag behind plasma insulin, (ii) fold difference in plasma versus saliva insulin levels, (iii) peak time for saliva insulin response post-meal, and (iv) time required for insulin levels to return to basal post-meal. In embodiments of the reference data set, each of these features could be characterized for different types of foods, such as both high-carbohydrate and low-carbohydrate meals.

Likewise, in another embodiment, the reference data set may be complied from comparisons of plasma and saliva insulin levels from individuals other than or in addition to the subject, who share a similar physiological condition or profile to the subject Similar to the reference data set for an individual subject, these reference data sets could be compiled from comparisons of plasma and saliva insulin levels at baseline and in response to different types of foodstuffs (high-carbohydrate and low-carbohydrate meals) at one or more different times post-meal. This may be used to generate insulin response profiles for individuals that share the physiological condition or profile, whereby the profile characterizes various features such as those identified above, namely: (i) saliva insulin lag behind plasma insulin, (ii) fold difference in plasma versus saliva insulin levels, (iii) peak time for saliva insulin response post-meal, and (iv) time required for insulin levels to return to basal post-meal. In embodiments of the reference data set based on physiological condition or profile, each of these features could likewise be characterized for different types of foods, such as both high-carbohydrate and low-carbohydrate meals.

As used herein, a “high-carborhydrate” refers to a foodstuff or meal that comprises at least 35% by weight carbohydrate. In a particular embodiment, a high-carbohydrate foodstuff or meal comprises at least 50% by weight carbohydrate. In an embodiment, a high-carbohydrate meal comprises by weight about 20% fat, about 55% carbohydrate and about 25% protein, and is about 500 kcal.

As used herein, a “low-carborhydrate” refers to a foodstuff or meal that comprises 25% or less carbohydrate by weight. In a particular embodiment, a low-carbohydrate foodstuff or meal comprises 15% or less carbohydrate by weight. In an embodiment, a low-carbohydrate meal comprises by weight about 65% fat, about 10% carbohydrate and about 25% protein, and is about 500 kcal.

Since the present invention establishes that levels of insulin in saliva correlate with levels of insulin in the blood after ingestion of different types of meals, comparing the level of insulin in saliva to the data reference set as described herein allows for determination of the subject's blood or plasma insulin level in respect of a particular foodstuff, e.g. a high-carbohydrate or a low-carbohydrate meal. From the one or more measured saliva insulin levels, the disclosed methods can thus be used to detect or predict a postprandial blood or plasma insulin response to the foodstuff.

As used herein, “postprandial blood or plasma insulin response” is intended to encompass not only the blood or plasma insulin level at a single time point post-meal (e.g. the time point corresponding to the measured saliva insulin level), but also the blood or plasma insulin levels over a time course post-meal. For instance, as disclosed herein, peak saliva insulin responses occur at about 60-90 minutes post-meal and return to basal at approximately 180 minutes for a high-carbohydrate meal. Thus, from one or more saliva insulin levels, the disclosed methods are capable of detecting or predicting the postprandial blood or plasma insulin level at time points corresponding to the one or more measured saliva insulin levels, as well as other times post-meal, adapted according to the type of foodstuff consumed. By “time point corresponding”, it is intended to take into account any lag between plasma insulin levels and saliva insulin levels.

In an embodiment, characterizing the subject's blood or plasma insulin level comprises multiplying the level of insulin in saliva by a factor of between about 2 to about 5. In an embodiment, characterizing the subject's blood or plasma insulin level comprises multiplying the level of insulin in saliva by a factor of about 2.0, about 2.5, about 3.0, about 3.5, about 4.0, about 4.5 or about 5. More particularly, in an embodiment, when the foodstuff is a high-carbohydrate meal the level of insulin in saliva is multiplied by a factor of about 4.0 (i.e. 4 times). Alternatively, in an embodiment in which the foodstuff is a low-carbohydrate meal the level of insulin in saliva is multiplied by a factor of about 2.0 (i.e. 2 times).

As disclosed in the examples herein, there is a lag time in the saliva insulin levels as compared to plasma insulin levels whereby saliva insulin levels tended to lag behind plasma insulin levels by about 30-60 minutes. Thus, in an embodiment, the step of characterizing the subject's blood or plasma insulin level comprises taking into account a lag time of about 30 minutes to about 60 minutes for the level of insulin in saliva. In an embodiment, the step of characterizing the subject's blood or plasma insulin level comprises taking into account a lag time of about 30 minutes, about 35 minutes, about 40 minutes, about 45 minutes, about 50 minutes, about 55 minutes or about 60 minutes for the level of insulin in saliva. In a particular embodiment, the step of characterizing the subject's blood or plasma insulin level comprises taking into account a lag time of about 30 minutes for the level of insulin in saliva.

Another aspect of the present invention relates to a method for determining whether a subject is in a fat-storage mode or a fat-burning mode, said method comprising: measuring a level of insulin in saliva from a subject; and comparing the level of insulin in saliva to a predetermined value to determine if the subject is in a fat-storage mode or a fat-burning mode, wherein a level of insulin in saliva above the predetermined value is indicative of a fat-storage mode and a level of insulin in saliva below the predetermined value is indicative of a fat-burning mode.

As used herein, the term “fat-storage mode” refers to a physiological status regarding fat metabolism in which the subject's body is in a state of inhibiting the breakdown of fat (e.g. triglycerides) within fat cells (i.e. adipose tissues) and/or stimulating the absorption of fatty acids and glucose from the blood to generate additional fat which is stored in the liver. Thus, the term “fat-storage mode” refers to a physiological state involving the inhibition of lipolysis (i.e fat breakdown) and/or an increase in lipogenesis (i.e. fat synthesis). It is known that insulin regulates fat metabolism and that high levels of inhibit lipolysis and promote lipogenesis. As used herein, “fat-storage mode” may be used interchangeably with “calorie-assimilation mode”.

As used herein, the term “fat-burning mode” refers to a physiological status regarding fat metabolism in which the subject's body is in a state of stimulating the breakdown of fat (e.g. triglycerides) within fat cells (i.e. adipose tissues) and/or inhibiting the absorption of fatty acids and glucose from the blood. Thus, the term “fat-burning mode” refers to a physiological state involving the stimulation of lipolysis (i.e fat breakdown) and/or the inhibition of lipogenesis (i.e. fat synthesis). It is known that insulin regulates fat metabolism and that low levels of promote lipolysis and inhibit lipogenesis. As used herein, “fat-burning mode” may be used interchangeably with “calorie-burning mode”.

In embodiments of the methods of determining whether a subject is in a fat-burning or fat-storage mode, the step of measuring a level of insulin in saliva from a subject may be performed in accordance with the techniques described elsewhere herein. For example and without limitation, in an embodiment the step of measuring the level of insulin in saliva may be performed by ELISA as described herein. Likewise, the methods may comprise a step of obtaining a saliva sample from a subject, which may be performed as described elsewhere herein.

The methods disclosed herein for determining whether a subject is in a fat-burning or fat-storage mode comprise comparing the level of insulin in saliva to a predetermined value. In an embodiment, the term “predetermined value” refers to the resting state saliva insulin level of an individual subject or a group of subjects that share a similar physiological condition or profile. By “resting state saliva insulin level”, it is meant the basal saliva insulin level at least four hours after the last consumption of a foodstuff and prior to ingestion of further foodstuff. In an embodiment, the basal saliva insulin level may be determined by one or more measurements of a subject's or a group of subjects' salivary insulin levels at least four hours after the last consumption of a foodstuff. In an embodiment, the resting state saliva insulin level is between about 0.5 mU/L and about 4.0 mU/L. In an embodiment, the resting state saliva insulin level is about 0.5 mU/L, about 1.0 mU/L, about 1.5 mU/L, about 2.0 mU/L, about 2.5 mU/L, about 3.0 mU/L, about 3.5 mU/L or about 4.0 mU/L.

As shown herein, the basal saliva insulin levels may be different for subjects classified as lean versus subjects classified as obese. As used herein, “lean”, “lean subject”, “lean individual” or “lean group” refer to a subject, individual or group of subjects who classify as normal weight (NW) based on the World Health Organization Guidelines (Consultation, 2008). These NW subjects have a BMI of 20.0-24.9 kg/m² with a waist-to-hip ratio of <0.90 for male or <0.85 for female. As used herein, “obese”, “overweight”, “obese subject”, “obese individual” or “obese group” refer to a subject, individual or group of subjects who classify as overweight/obese (OO) based on the World Health Organization Guidelines (Consultation, 2008). These OO subjects have a BMI of ≥28.0 kg/m² with a waist-to-hip ratio of ≥0.90 for male or ≥0.85 for female.

In an embodiment, the resting state saliva insulin level value for a lean subject is between about 1.0 mU/L and about 2.0 mU/L of salivary insulin. In an embodiment, the resting state saliva insulin level for a lean subject is about 1.0 mU/L, about 1.1 mU/L, about 1.2 mU/L, about 1.3 mU/L, about 1.4 mU/L, about 1.5 mU/L, about 1.6 mU/L, about 1.7 mU/L, about 1.8 mU/L, about 1.9 mU/L or about 2.0 mU/L of salivary insulin. In an embodiment, the resting state saliva insulin level for an obese subject is between about 2.0 mU/L and about 3.0 mU/L of salivary insulin. In an embodiment, the resting state saliva insulin level for an obese subject is about 2.0 mU/L, about 2.1 mU/L, about 2.2 mU/L, about 2.3 mU/L, about 2.4 mU/L, about 2.5 mU/L, about 2.6 mU/L, about 2.7 mU/L, about 2.8 mU/L, about 2.9 mU/L or about 3.0 mU/L of salivary insulin.

In another embodiment, the term “predetermined value” refers to the resting blood or plasma insulin level of an individual subject or a group of subjects that share a similar physiological condition or profile. By “resting state blood or plasma insulin level”, it is meant the basal blood or plasma insulin level at least four hours after the last consumption of a foodstuff and prior to ingestion of further foodstuff. In an embodiment, the basal blood or plasma insulin level may be determined by one or more measurements of a subject's or a group of subjects' blood or plasma insulin levels at least four hours after the last consumption of a foodstuff. In an embodiment, the resting state blood or plasma insulin level is between about 4.0 mU/L and about 8.0 mU/L. In an embodiment, the resting state blood or plasma insulin level is about 4.0 mU/L, about 4.5 mU/L, about 5.0 mU/L, about 5.5 mU/L, about 6.0 mU/L, about 6.5 mU/L, about 7.0 mU/L, about 7.5 mU/L or about 8.0 mU/L.

As shown herein, the basal blood or plasma insulin levels may be different for subjects classified as lean versus subjects classified as obese. In an embodiment, the resting state blood or plasma insulin level value for a lean subject is between about 4.0 mU/L and about 6.0 mU/L of blood or plasma insulin. In an embodiment, the resting state blood or plasma insulin level for a lean subject is about 4.0 mU/L, about 4.1 mU/L, about 4.2 mU/L, about 4.3 mU/L, about 4.4 mU/L, about 4.5 mU/L, about 4.6 mU/L, about 4.7 mU/L, about 4.8 mU/L, about 4.9 mU/L, about 5.0 mU/L, about 5.1 mU/L, about 5.2 mU/L, about 5.3 mU/L, about 5.4 mU/L, about 5.5 mU/L, about 5.6 mU/L, about 5.7 mU/L, about 5.8 mU/L, about 5.9 mU/L or about 6.0 mU/L of blood or plasma insulin. In an embodiment, the resting state blood or plasma insulin level for an obese subject is between about 6.0 mU/L and about 8.0 mU/L of blood or plasma insulin. In an embodiment, the resting state blood or plasma insulin level for an obese subject is about 6.0 mU/L, about 6.1 mU/L, about 6.2 mU/L, about 6.3 mU/L, about 6.4 mU/L, about 6.5 mU/L, about 6.6 mU/L, about 6.7 mU/L, about 6.8 mU/L, about 6.9 mU/L, about 7.0 mU/L, about 7.1 mU/L, about 7.2 mU/L, about 7.3 mU/L, about 7.4 mU/L, about 7.5 mU/L, about 7.6 mU/L, about 7.7 mU/L, about 7.8 mU/L, about 7.9 mU/L or about 8.0 mU/L of blood or plasma insulin.

As will be appreciated, the predetermined value is a discrete value, but one which is different depending upon the resting state insulin level of an individual subject or the resting state insulin level of a group of subjects that share a similar physiological condition or profile. It is within the ability of the skilled person to determine the predetermined value as would be relevant to any particular subject of the methods of the present invention. For example, if the subject is one that would be classified as lean, a predetermined value for lean subjects may be used, such as without limitation those set forth above. Likewise, if the subject is one that would be classified as obese, a predetermined value for obese subjects may be used, such as without limitation those set forth above.

In the methods disclosed herein for determining whether a subject is in a fat-burning or fat-storage mode, a level of insulin above the predetermined value is indicative of a fat-storage mode and a level of insulin in saliva below the predetermined value is indicative of a fat-burning mode. Since the methods involve measuring the level of insulin in saliva, the measured level may be directly compared to the predetermined value being the resting state saliva insulin level. Alternatively, the disclosed methods may further comprise a step of characterizing the subject's blood or plasma insulin level based upon the level of insulin in saliva. This step may be performed as described elsewhere herein in relation to embodiments of the methods for detecting or predicting a postprandial blood or plasma insulin response to a foodstuff. In embodiments in which this additional step is performed, the determined level of insulin in blood or plasma may be compared to the predetermined value being the resting state blood or plasma insulin level.

The method of determining whether a subject is in a fat-burning or fat-storage mode may advantageously be performed after ingestion of a foodstuff, such as a high-carbohydrate meal and/or a low-carbohydrate meal. As disclosed herein, it was found that saliva insulin responses were able to delineate between low and high insulin responses to different meals throughout the day in both lean and obese individuals. Accordingly, the method disclosed herein may be advantageously applied to determine whether any particular foodstuff or meal would result in (or maintain) the subject's body being in a state of fat-burning or fat-storage. The disclosed methods may also be used to predict the outcome of other foodstuffs of a similar composition (both high carbohydrate or both low carbohydrate) or an alternate composition (high carbohydrate versus low carbohydrate, and vice versa) on the subject's fat metabolism status (i.e. fat-burning or fat-storage). For subjects on a diet or for those that are athletes, this may be of particular and practical advantage in determining the foodstuffs that can be consumed at certain times of day, and avoid those which should not be consumed at such times depending on the desired outcome of a fat-burning or fat-storage mode.

Another embodiment of the present invention relates to a method for diagnosing and/or monitoring hyperinsulinemia in a subject, said method comprising measuring a level of insulin in saliva from a subject following ingestion of a high carbohydrate meal and/or a low-carbohydrate meal.

Hyperinsulinemia is a condition in which there are excess levels of insulin circulating in the blood relative to the level of glucose. Hyperinsulinemia is often seen in people with early stage type 2 diabetes (prediabetes). Hyperinsulinemia is frequently associated with hypertension, obesity, dyslipidemia and glucose intolerance, and these conditions are collectively known as Metabolic Syndrome. Prior methods for the diagnosis of hyperinsulinemia typically involve observation of fasting and postprandial blood or plasma insulin levels with a normal meal or with 100 grams of oral glucose.

The present invention provides alternative and advantageous methods of diagnosing and/or monitoring hyperinsulinemia based on the finding, for example, that saliva insulin levels are able to delineate between low and high insulin responses to different types of meals (i.e. insulin levels in saliva correlate with levels of insulin in the blood after eating different types of foodstuffs).

As used herein, the terms “diagnosing” and “diagnose” interchangeably refer to the ability to identify the nature of an illness or problem, i.e. as being hyperinsulinemia. The terms “monitoring” or “monitor” are used interchangeably herein to refer to the ability to track the course and/or seriousness of an illness or problem over a period of time, i.e. to track the progression of hyperinsulinemia. Monitoring also encompasses the ability to track the effectiveness of a particular treatment for hyperinsulinemia by use of the disclosed methods.

The methods disclosed herein for diagnosing and/or monitoring hyperinsulinemia in a subject comprise measuring a level of insulin in saliva from a subject. This step may be performed in accordance with the techniques described elsewhere herein. For example and without limitation, in an embodiment the step of measuring the level of insulin in saliva may be performed by ELISA as described herein. Likewise, the methods may comprise a step of obtaining a saliva sample from a subject, which may be performed as described elsewhere herein.

The level of insulin in saliva is measured following ingestion of a high-carbohydrate and/or a low-carbohydrate meal. From the measured value, and based on the disclosure herein that insulin levels in saliva correlate with levels in the blood, it can be determined whether a subject has or is likely to have hyperinsulinemia. For example, an abnormally high saliva insulin level in response to a high-carbohydrate meal or low-carbohydrate meal would be indicative of hyperinsulinemia. By “abnormally high”, it is meant that the saliva insulin level is elevated as compared to the value for a normal subject who does not have hyperinsulinemia. An abnormally high level may also be identified by characterizing the blood or plasma insulin level from the saliva insulin level (as described elsewhere herein), and determining if the blood or plasma insulin is elevated as compared to the value for a normal subject who does not have hypersinsulinemia. Generally, obese subjects may already have at least some degree of hyperinsulinemia and thus, in an embodiment, the normal subject is a lean subject.

In an embodiment, an abnormally high saliva insulin level after a high-carbohydrate meal is a level above about 6 mU/L of saliva insulin. In an embodiment, an abnormally high saliva insulin level after a high-carbohydrate meal is about 6 mU/L, about 7 mU/L, about 8 mU/L, about 9 mU/L, about 10 mU/L, about 11 mU/L, about 12 mU/L, about 13 mU/L, about 14 mU/L, about 15 mU/L, about 16 mU/L, about 17 mU/L, about 18 mU/L, about 19 mU/L, about 20 mU/L or more of saliva insulin.

In an embodiment, an abnormally high blood or plasma insulin level after a high-carbohydrate meal is a level above about 50 mU/L of blood or plasma insulin. In an embodiment, an abnormally high blood or plasma insulin level after a high-carbohydrate meal is about 50 mU/L, about 55 mU/L, about 60 mU/L, about 65 mU/L, about 70 mU/L, about 75 mU/L, about 80 mU/L, about 85 mU/L, about 90 mU/L, about 95 mU/L, about 100 mU/L, about 105 mU/L, about 110 mU/L, about 115 mU/L, about 120 mU/L, about 125 mU/L, about 130 mU/L, about 135 mU/L, about 140 mU/L, about 145 mU/L, about 150 mU/L or more of blood or plasma insulin.

In an embodiment, an abnormally high saliva insulin level after a low-carbohydrate meal is a level above about 3 mU/L of saliva insulin. In an embodiment, an abnormally high saliva insulin level after a low-carbohydrate meal is about 3 mU/L, about 3.5 mU/L, about 4 mU/L, about 4.5 mU/L, about 5 mU/L, about 5.5 mU/L, about 6 mU/L, about 6.5 mU/L, about 7 mU/L, about 7.5 mU/L, about 8 mU/L, about 8.5 mU/L, about 9 mU/L, about 9.5 mU/L, about 10 mU/L or more of saliva insulin.

In an embodiment, an abnormally high blood or plasma insulin level after a low-carbohydrate meal is a level above about 10 mU/L of blood or plasma insulin. In an embodiment, an abnormally high blood or plasma insulin level after a low-carbohydrate meal is about 10 mU/L, about 15 mU/L, about 20 mU/L, about 25 mU/L, about 30 mU/L, about 35 mU/L, about 40 mU/L, about 45 mU/L, about 50 mU/L or more of blood or plasma insulin.

In an embodiment, diagnosis of hyperinsulinemia may also comprise measuring the subject's fasting or resting state saliva insulin level. Again, an abnormally high level as compared to a normal subject would be indicative of hyperinsulinemia, particularly in combination with an even more elevated saliva insulin level or blood/plasma insulin level after ingestion of a foodstuff. In an embodiment, an abnormally high level of resting state saliva insulin is a level above about 2 mU/L. In an embodiment, an abnormally high resting state saliva insulin level is about 2 mU/L, about 2.5 mU/L, about 3 mU/L, about 3.5 mU/L, about 4 mU/L, about 4.5 mU/L, about 5 mU/L or more of saliva insulin.

An abnormally high resting state level may also be identified by characterizing the blood or plasma insulin level from the resting state saliva insulin level (as described elsewhere herein), and determining if the blood or plasma resting state insulin is elevated as compared to the value for a normal subject who does not have hypersinsulinemia. In an embodiment, an abnormally high level of resting state saliva insulin is a level above about 6 mU/L. In an embodiment, an abnormally high resting state saliva insulin level is about 6 mU/L, about 6.5 mU/L, about 7 mU/L, about 7.5 mU/L, about 8 mU/L, about 8.5 mU/L, about 9 mU/L, about 9.5 mU/L, about 10 mU/L, or more of blood or plasma insulin.

In another embodiment, the methods of diagnosing or monitoring hyperinsulinemia may comprise comparing the level of insulin in saliva to a reference data set (as described elsewhere herein). The reference data set may be compiled based on data from an individual subject or based on data from a group of subjects that share a similar physiological condition or profile.

In an embodiment, the reference data set may comprise historical data for the specific subject, e.g. data from prior to the development of hyperinsulinemia or data from earlier in the progression of hyperinsulinemia. By comparing to this data, it is possible to observe changes in insulin responses over time and thereby diagnose and/or monitor hyperinsulinemia in the subject.

In another embodiment, the reference data set may comprise data from a group of subjects other than or in addition to the subject that provides a representation of a normal insulin response for subject of that physiological condition or profile. By comparing to this data, it is possible to observe differences in the subject's insulin responses as compared to the norm for their physiological condition or profile and thereby diagnose and/or monitor hyperinsulinemia in the subject.

In embodiments of the methods for diagnosing and/or monitoring hyperinsulinemia, measuring the level of insulin in saliva is performed both after consumption of both a high-carbohydrate meal and a low-carbohydrate meal. By examining both of these levels, it will be appreciated that a more accurate diagnosis of hyperinsulinemia could potentially be made.

The following examples are provided to more fully describe the disclosure and are presented for non-limiting illustrative purposes.

### EXAMPLES

### Example 1

A study was performed with two groups of 8 individuals per group. The first group consisted of lean individuals and the second group consisted of individuals considered to be “overweight/obese”. The inclusion criteria for the test individuals included (i) between 20-40 years old, (ii) free of any disease or illness symptoms, (iii) no prescribed medications, and (iv) not on a vegetarian diet or a low-carbohydrate diet. Additionally, the following anthropometric criteria were used in the selection process: (i) the “lean” individuals had to have a body mass index between 20-24.9 kg/m² with a waist-to-hip ratio less than 0.90 male or <0.85 female, and (ii) the “overweight/obese” individuals had to have a body mass index equal to or greater than 28.0 kg/m² with a waist-to-hip ratio equal to or greater than 0.90 male or 0.85 female.

The primary objectives of this study were to: (i) determine if salivary insulin can be used to delineate between low and high insulin levels following the ingestion of low- and high-carbohydrate mixed meals, and (ii) compare young lean participants to young overweight/obese participants to determine if subtle differences in postprandial hyperinsulinemia could be detected in saliva.

### Participants

Sixteen individuals were recruited through poster advertisement and word of mouth across the University of British Columbia Okanagan campus. Based on the World Health Organization (WHO) guidelines (Consultation, 2008), eight individuals were classified as normal weight (NW) (BMI 20.0-24.9 kg/m² with a waist to hip ratio <0.90 male or <0.85 female) and eight classified as overweight/obese (OO) (BMI ≥28.0 kg/m² with a waist to hip ratio ≥0.90 male or ≥0.85 female). All participants met the following eligibility criteria: (1) being between 20 and 39 years of age; (2) not diagnosed with any medical conditions; (3) not taking any medications known to impact metabolism (on stable oral contraceptive pills for at least 3 months was accepted) (4) not following a vegetarian or low-carbohydrate high-fat diet; (5) not a competitive athlete or participating in structured endurance training The study was approved by the UBC Clinical Research Ethics Board and registered at ClinicalTrials.gov (NCT02699203). Participants' baseline characteristics are summarized in Table 1.

TABLE 1
Characteristics of participants

| | Lean | Overweight/obese |
|---|---|---|
| Number of individuals | 8 | 7 |
| Gender (M/F) | 5/3 | 6/1 |
| Age (yrs) | 27.1 ± 4.1 | 30.6 ± 4.3 |
| Body mass index (kg/m²) | 22.4 ± 1.8 | 32.1 ± 1.8 |
| Waist-to-hip ratio (cm) | 0.79 ± 0.06 | 0.93 ± 0.06 |
| Systolic blood pressure (mmHg) | 118 ± 7 | 129 ± 12 |
| Diastolic blood pressure (mmHg) | 76 ± 8 | 83 ± 7 |

### Study Design

The study followed a randomized crossover design. Randomization was performed using the online research randomizer program accessible at: https://www.randomizer.org. Eligible participants completed two isocaloric meal conditions separated by at least 72 hours: (1) low-carbohydrate meal and (2) high-carbohydrate meal.

### Study Protocol

Visit 1: After the eligibility criteria were confirmed and informed consent obtained, anthropometrics and blood pressure measurements were collected. Participants were given a dietary journal to record all the food and drinks consumed for the 24-hour prior to their first experimental condition. During this 24-hour period, participants were instructed not to exercise and to follow their typical eating patterns such that replication would be easily accomplished on the day preceding their second experimental condition. Visit 1 occurred 2-10 days prior to Visit 2.

Visits 2 and 3: After an overnight (>10 h) fast, participants arrived at the laboratory where the research coordinator reviewed the dietary journal and confirmed that no exercise was performed in the previous 24 hours. If there were no irregularities an indwelling venous catheter was inserted by a certified phlebotomist in the antecubital space of the arm. Fasting blood and saliva samples were then collected followed by the consumption of the meal, which was consumed within 10 minutes. Five minutes before the second sampling time point participants were asked to rinse their mouth with water to remove any food remnants Blood and saliva samples were then collected at 15, 30, 60, 90 and 120 minutes following meal completion. Following visit 2 participants were provided with their 24-hour diet record and given instructions to follow their meal plan exactly prior to the next visit. On the morning of visit 3 (3-10 days following visit 2) the research coordinator reviewed the 24-hour dietary journal for compliance, and confirmed that no exercise had been performed on the day before. Participants then went through the same procedures as visit 2 but consumed the alternate meal.

### Meals

The low-carbohydrate meal (10% carbohydrate, 65% fat, 25% protein) was composed of whole eggs, egg whites, avocado, red peppers and onions. The high-carbohydrate meal (55% carbohydrate, 20% fat, 25% protein, Glycemic index: 48) was composed of plain rolled oats, mixed berries (blueberries, raspberries, strawberries) and stevia sweetened whey protein isolate. Both mixed meals were isocaloric (500 kcal) and were designed to reflect food typically eaten at breakfast that would elicit a low and high insulin response, respectively.

### Blood and Saliva Sample Collection and Processing

Repeated blood samples were collected in 4 ml EDTA tubes (BD Vacutainer, Franklin Lakes, N.J., USA) using an intravenous catheter (BD Nexiva, Sandy, Utah, USA). Saliva samples were collected using a passive drool collection device for a period of 60 seconds (Salimetrics LLC, State College, Pa., USA). Both samples at each corresponding time point were kept on ice and then centrifuged together within 20 minutes (1550 g, 15 minutes, 4° Celsius). Plasma was immediately stored with the centrifuged saliva samples at −20° C. prior to analyses. For saliva analyses, samples were first thawed and then centrifuged again (1550 g, 15 minutes, 4° C.). The clarified supernatant used for insulin analysis.

### Biochemical Analyses

Plasma glucose was measured by the hexokinase method on a clinical chemistry analyzer (Chemwell 2910, Awareness Technologies). Plasma and salivary insulin were measured in duplicate by ELISA following the manufacturer's protocol (Mercodia Ultrasensitive Insulin ELISA) with absorbance read on a microplate reader (iMark, Bio-Rad). The coefficient of variation for duplicate samples was 10.7% for plasma insulin and 6.0% for salivary insulin. Saliva was diluted 1:1 with the zero standard from the kit based on optimization experiments showing inference in spike and recovery testing when neat saliva was used in the assay. Recovery of spiked insulin in 1:1 diluted saliva was >80%.

### Statistics

Data was analyzed using SPSS v22. Normality was assessed using Q-Q plots and Shapiro-Wilk test within each group. Appropriate transformation (natural log or 1/square root) on non-normally distributed variables resulted in normal distribution. Baseline differences were assessed using an unpaired Student t-test. Area under the curve (AUC) and incremental AUC (iAUC) were calculated using GraphPad Prism v6.0. A two factor (group X meal) ANOVA with repeated measures on the second factor was used to analyze AUC and iAUC for plasma glucose, plasma insulin, and saliva insulin. Significant interactions were followed up with pre-planned contrasts comparing lean to obese within meal and low-carbohydrate to high-carbohydrate meals within groups using Bonferroni corrections for multiple comparisons. Potential relationships between salivary and plasma iAUC following both meals were assessed using Pearson correlations. Significance was set at P<0.05.

### Results

All participants complied with replication of their diet and refrained from exercising for the 24-hour period preceding each experimental condition.

Baseline characteristics are presented in Table 1. As expected, the group classified as overweight or obese had a higher body mass index (BMI) and waist to hip ratio (WHR) (both p<0.001). The OO group also had a significantly higher systolic blood pressure, fasting blood glucose, fasting plasma insulin and fasting salivary insulin (all p<0.05). There were no differences between the two groups in terms of age, resting heart rate and diastolic blood pressure.

The saliva and plasma insulin responses to the meals for both groups are presented in FIGS. 1A-1D. It can be seen that for both plasma and saliva, the HC meal leads to a larger insulin response and that individuals with obesity have an overall higher response than lean counterparts to both meals.

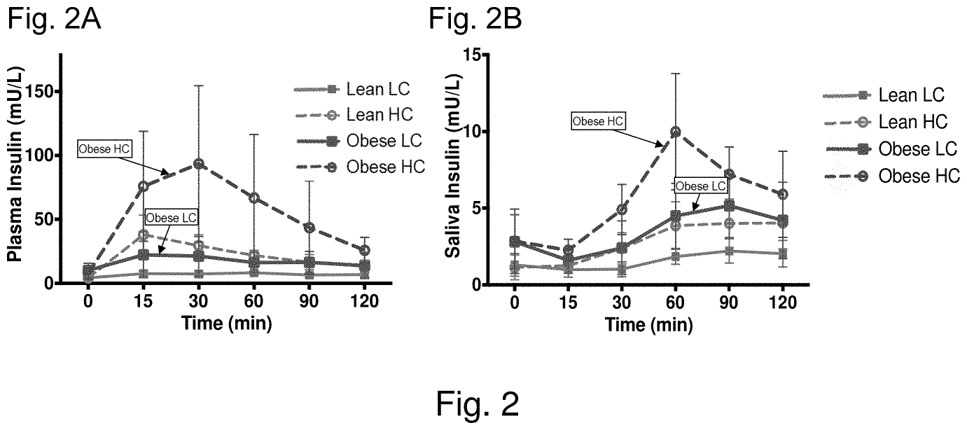

In FIGS. 2A (plasma insulin) and 2B (saliva insulin), the data is shown together for both meals and both subgroups. The data in FIG. 2A show that plasma insulin levels were elevated in both the lean subgroup and the obese subgroup receiving the high-carbohydrate diet within 15 minutes of completion of the meal, peaked at around 15-30 minutes and then returned to near normal after 120 min. The data in FIG. 2B show that saliva insulin levels were relatively unchanged for the first 15 minutes after completion of both meals by both subgroups, but then increased after 30 minutes and peaked at 60 minutes after completion of the meals. The data in FIG. 2B indicate that elevated levels of insulin persisted for more than 2 hr after the meals were completed.

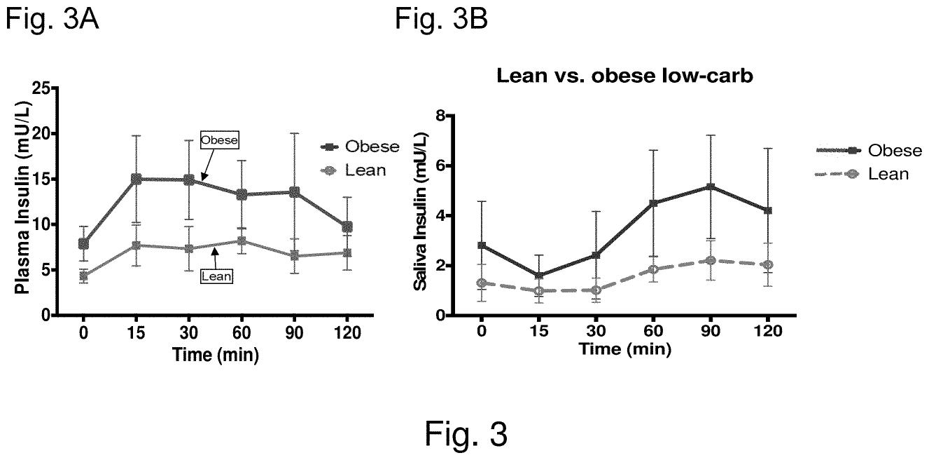

The data in FIG. 3A show that plasma insulin levels were somewhat elevated in both lean and obese subgroups receiving the low-carbohydrate meal. FIG. 3B shows that while the saliva insulin did not vary significantly in the lean group receiving the low-carbohydrate meal, increased levels of saliva insulin were detected in the obese group at 60 min, 90 min, and 120 min after completion of the low-carbohydrate meal.

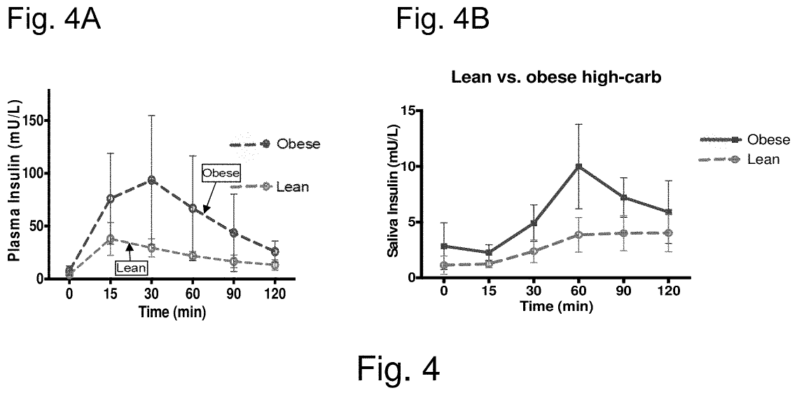

The data in FIG. 4A show that the lean subgroup receiving the high-carbohydrate meal evidenced elevated plasma insulin after 15 min, and the elevated levels quickly dropped during the subsequent sampling time. The obese subgroup showed quickly increasing plasma insulin levels at the 15 min and 30 min sampling times, after which their plasma insulin levels fell to near normal levels after 120 min. However, the saliva insulin levels increased in both lean and obese subgroups after 30 min (FIG. 4B).

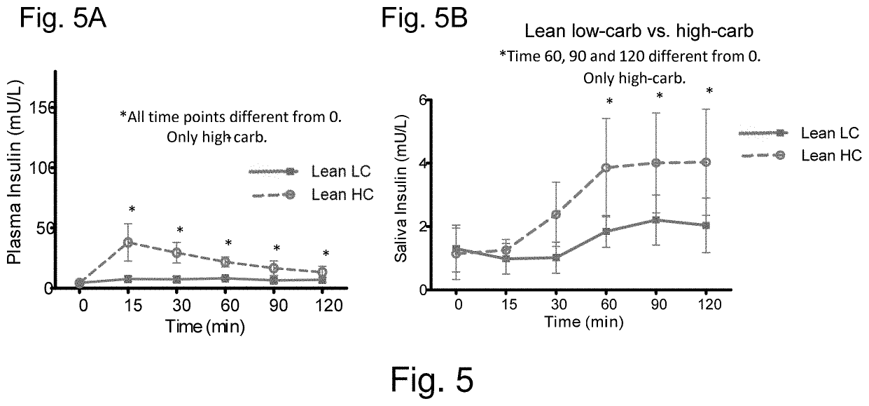

FIG. 5A shows that the lean subgroup receiving the high-carbohydrate meal showed elevated levels of plasma insulin after 15 but the levels dropped for the subsequent sampling periods. It appeared that the lean subgroup receiving the low-carbohydrate did not show any changes in their plasma insulin for the 2-hour post-consumption period. However, FIG. 5B shows that the lean subgroup receiving the high-carbohydrate meal evidenced elevated levels of saliva insulin after 30 min and further increases after 60 min, whereafter the saliva insulin level remained elevated.

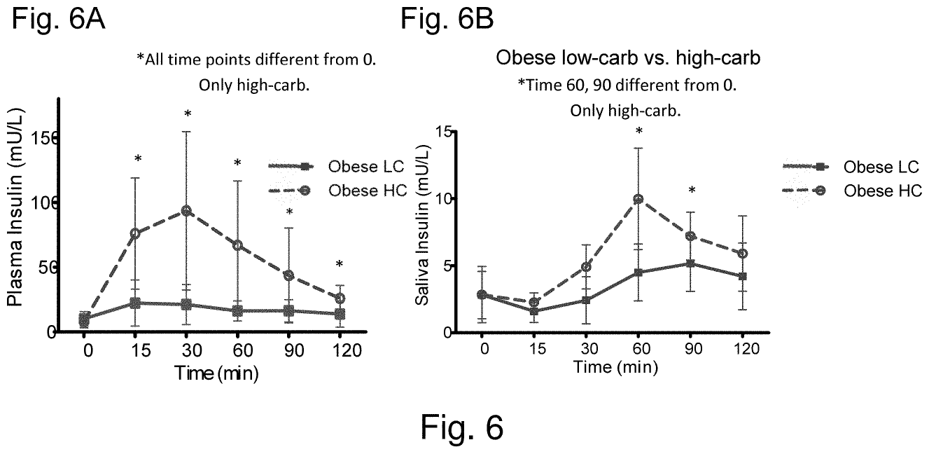

FIG. 6A shows that the plasma insulin levels in the obese subgroup receiving the high-carbohydrate meal increased for the first 30 min post-consumption and then fell to normal levels by the end of the 120-min period. However, the obese group that received the low-carbohydrate meal did not evidence any significant changes in their plasma insulin levels over the 120-min sampling period. FIG. 6B shows that the saliva insulin levels increased with both types of meals for the first 60 min post-consumption and then fell.

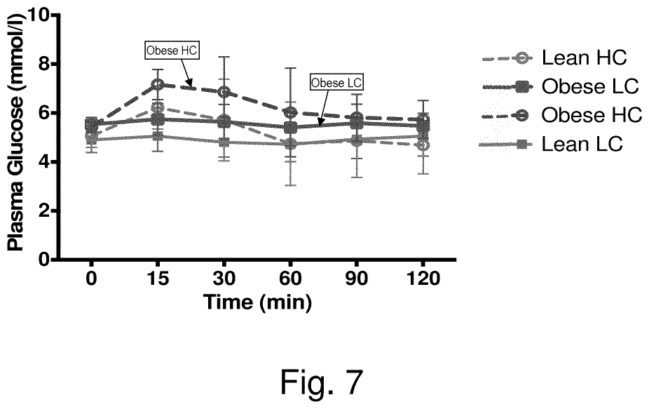

The data in FIG. 7 show that neither type of carbohydrate meal caused significant changes in the plasma glucose levels in all four subgroups over the 120-min sampling period.

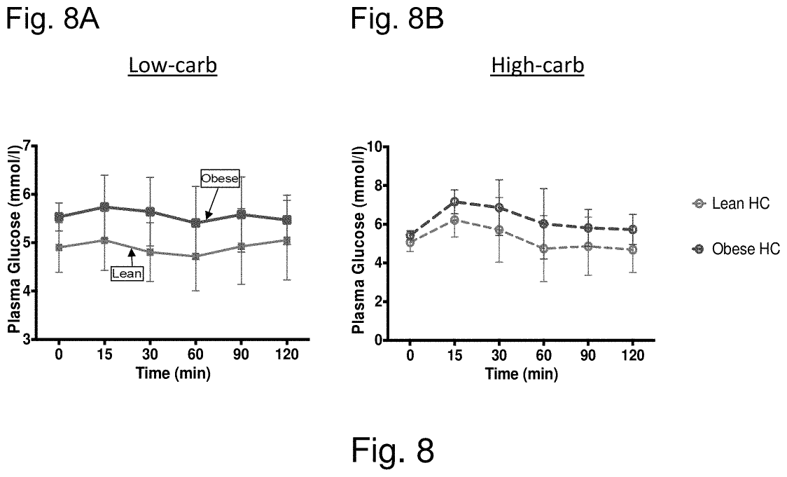

The data in FIGS. 8A and 8B show that although plasma glucose levels were elevated in the obese subgroups receiving both types of carbohydrate meals compared to the plasma glucose levels in the lean subgroups, blood glucose levels did not fluctuate significantly over the 120-min sampling period in any of the four subgroups.

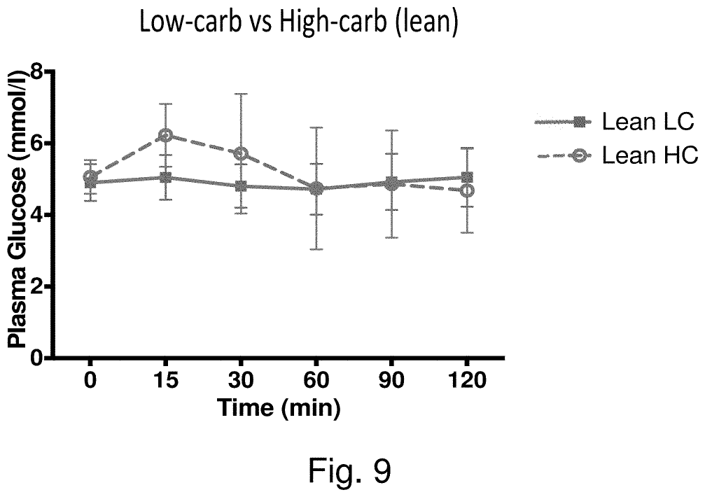

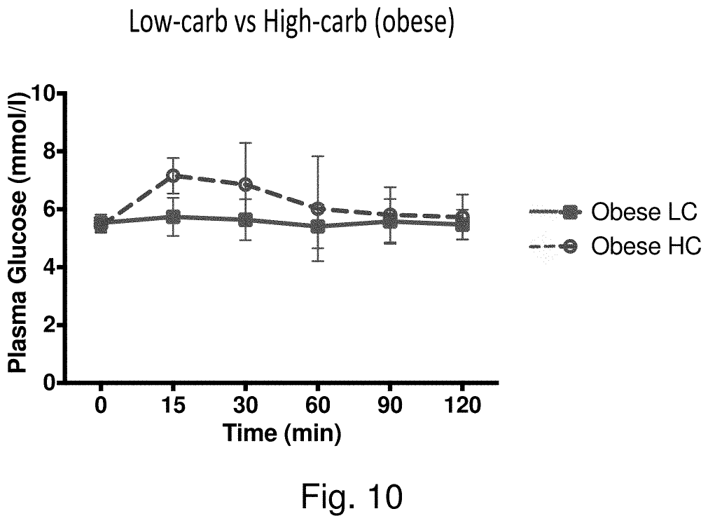

The data in FIG. 9 show that while the lean subgroup receiving the high-carbohydrate meal had slightly elevated plasma glucose compared to the lean subgroup receiving the low-carbohydrate meal for the first 30 min post-consumption, the levels were approximately the same for the subsequent duration of the sampling periods. Likewise, the data in FIG. 10 show that while the obese subgroup receiving the high-carbohydrate meal had slightly elevated plasma glucose compared to the obese subgroup receiving the low-carbohydrate meal for the first 30 min post-consumption, the levels were approximately the same for the subsequent duration of the sampling periods.

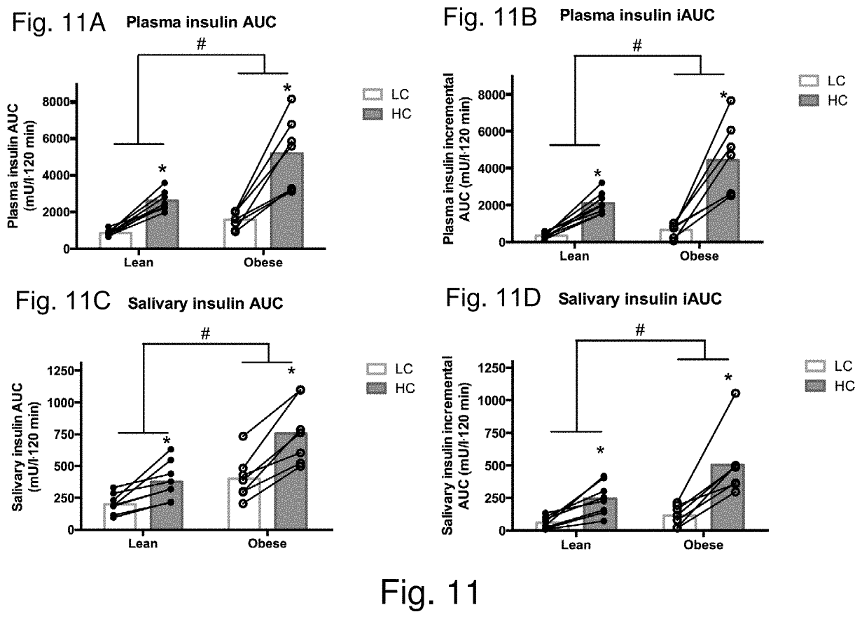

Total and incremental AUC are shown in FIGS. 11A-11D. Significant Meal X Group interactions were observed for salivary insulin AUC (p=0.025), plasma insulin AUC (p=0.014), salivary insulin iAUC (p=0.036) plasma insulin iAUC (p=0.015). In the lean group, salivary insulin AUC (by ˜89%; p=0.005), plasma insulin AUC (by ˜205%; p<0.001), salivary insulin iAUC (by ˜307%; p=0.002) and plasma insulin iAUC (by ˜519%; p<0.001) were higher after HC as compared to LC. In the obese group, salivary insulin AUC (by ˜90%; p=0.001), plasma insulin AUC (by ˜230%, p=0.002) salivary insulin iAUC (by ˜340%; p=0.003) and plasma insulin iAUC (by ˜582%; p=0.002) were also higher after HC as compared to LC. Salivary insulin AUC (by ˜100%, p=0.003), plasma insulin AUC (by ˜98%, p=0.014) salivary insulin iAUC (by ˜106%, p=0.022) and plasma insulin iAUC (by ˜111%, p=0.020) were significantly higher in the OO group as compared to the NW group after the HC breakfast. After the LC breakfast, salivary insulin AUC (p=0.010) and plasma insulin AUC (p=0.007) were significantly higher by ˜100% and ˜83%, respectively, in the OO group as compared to the NW group. Plasma insulin iAUC and salivary insulin iAUC, despite being higher in the OO group compared to the NW group after LC, did not reach statistical significance (respectively p=0.067 and p=0.119).

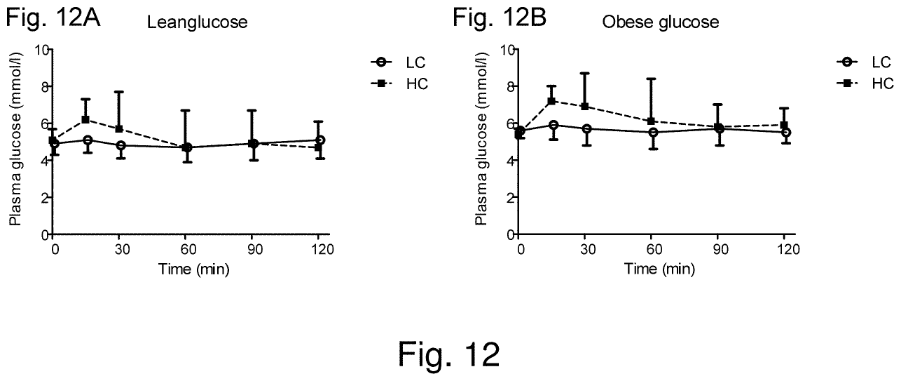

The plasma glucose responses to the meals for both groups are shown in FIGS. 12A-12B. No Meal X Group interactions were found for plasma glucose AUC (p=0.436) (NW; HC: 618±189 vs LC: 586±94, OO; HC: 751±156 vs LC: 678±94) or plasma glucose iAUC (p=0.261) (NW; HC: 64±108 vs LC: 21±32, OO; HC: 117±118 vs LC: 33±42). The main effect of meal for plasma glucose AUC approached statistical significance (HC; 680±182 vs LC; 629±102, p=0.057). However, there was a significant main effect of meal for plasma glucose iAUC (HC; 89±112 vs LC; 27±36, p=0.012) indicating higher values after the HC meal compared to the LC meal. There were no significant differences between the NW or OO groups for plasma glucose AUC (p=0.296) and iAUC (p=0.122).

Relationships between plasma and saliva insulin are shown in FIGS. 13A-13C. The fasting salivary:plasma insulin ratio was 1:3.6 in NW and 1:2.8 in OO. Fasting plasma insulin and fasting salivary insulin showed a significant positive correlation (r=0.602, p=0.017). Plasma insulin AUC was significantly correlated with salivary insulin AUC after the LC meal (r=0.821; p<0.000) and the HC meal (r=0.882; p<0.000). Plasma insulin iAUC was significantly correlated with salivary insulin iAUC after the HC meal (r=0.731; p=0.002) but not the LC meal (r=0.126; p=0.654). Saliva flow rate over 60 seconds was not significantly different across each time points or between meals and groups (data not shown). The peak salivary and plasma insulin values are presented in Table 2.

TABLE 2
Resting levels of salivary insulin, plasma insulin, and plasma
glucose after completion of the fasting period, just prior
to food consumption for the lean group and the obese group.

| | lean | Overweight/obese | P value* |
|---|---|---|---|
| Salivary insulin (mU/L) | 1.22 ± 0.90 | 2.83 ± 2.02 | 0.046* |
| Plasma insulin (mU/L) | 4.29 ± 0.66 | 6.95 ± 1.55 | 0.003* |
| Plasm glucose (mmol/L) | 5.0 ± 0.5 | 5.5 ± 0.3 | 0.041* |

The data generated during this study demonstrate that testing for saliva insulin levels at various times after food consumption can be a useful tool for detecting an individual's metabolic condition with regard to whether they are in a fat-storing mode or in a fat-burning mode and, therefore, can be used to monitor and manage food consumption with respect to e.g. weight management and fitness goals.

Specifically, the data shows that: i) fasting saliva insulin positively correlates to fasting plasma insulin, indicating that saliva insulin could be used to identify those with baseline insulin resistance (higher fasting insulin); ii) saliva insulin profiles following a high-carbohydrate breakfast are significantly higher than saliva insulin profiles after a low-carbohydrate breakfast in a similar manner to the expected response based on plasma; iii) overweight/obese individuals have a higher baseline and post-breakfast saliva insulin response to both high- and low-carbohydrate breakfast meals, similar to responses seen for plasma. It was also found that the saliva insulin response tends to lag behind plasma insulin by about 30-60 minutes and that peak saliva insulin responses occurred about 60-90 minutes following a high-carbohydrate meal, although there were some individual differences between participants.

Altogether, these data provide evidence that saliva insulin could be used to track or monitor low or high insulin levels in order to provide an accurate representation of an individuals' response to foods, state of insulin resistance and/or metabolic health. This data has been published in Myette-Côté 2017, which is incorporated in its entirety herein by reference.

### Example 2

In Example 1 (also published as Myette-Cote, 2017), it was shown that saliva insulin responses were able to delineate between low and high insulin responses to breakfast meals in lean and overweight/obese individuals.

In this example, insulin responses were studied following a meal other than breakfast. This is important because it was noted in Example 1 that saliva insulin did not appear to return to baseline levels within 120 min following meal consumption and that the return to baseline saliva insulin appeared to lag behind plasma insulin. In order for an individual to track their insulin levels to monitor food responses or insulin resistance status, it would be useful to know at what time of day (or what meal) is most conducive to assessing saliva insulin. Furthermore, this study examined the impact of physical activity/exercise on saliva insulin responses.

In order to address these issues, the study in this example was undertaken to explore saliva insulin profiles in response to three meals consumed every three hours across a nine-hour laboratory visit. To explore how physical activity/exercise influences saliva (and plasma) insulin responses, a model was employed whereby short activity breaks were interspersed throughout prolonged sitting. This model was chosen because recent studies report that insulin and glucose control is improved (i.e. lower) when participants break up prolonged sitting with brief activity breaks and it would allow for feasible, standardized application of exercise at multiple times throughout the day.

### Study Design & Protocol

Schematic 1 depicts the experimental protocol. Eight healthy, young males (age: 18-35, BMI=18.5-25.0) and four adults with elevated waist circumference (age 18-65; with a waist circumference of 102 centimeters (40 inches) or more in men, or 88 centimeters (34 inches) or more in women) reported to the laboratory following an overnight fast on three separate occasions in a randomized crossover design. The experimental conditions were: high-carbohydrate seated (HC), high-carbohydrate active (ACT), and low-carbohydrate seated (LC). Physical activity was avoided and diet was recorded and replicated on the day before each trial. Upon arrival at the laboratory, an indwelling venous catheter was inserted into an antecubital vein and a baseline fasting blood sample was obtained. Participants then provided a baseline saliva sample (passive drool) and consumed the breakfast meal. Repeat blood and saliva samples were obtained every 30 minutes across 540 minutes (9 hours). A lunch meal was consumed at 180 minutes and dinner meal consumed at 360 minutes (i.e., meal consumed every 180 minutes/3 hours). In the HC and LC conditions, the participants remained seated for the entire protocol. In the ACT condition, participants remained seated except that every 60 minutes they performed a 20-second stair climb at a vigorous pace. Notably, some participants with elevated waist circumference took up to 60 seconds to complete the stair climb. Short stair climbs were used because they involve a large muscle mass (to improve insulin sensitivity), were feasible to repeat across the day when compared to multiple longer exercise bouts, and are easily translatable to real-life.

Saliva and plasma insulin were assessed by standard ELISA (Mercodia Ultrasensitive Human Insulin ELISA for saliva, Crystal Chem Human Insulin ELISA for plasma). Our previous work showed that the Mercodia assay can measure insulin in saliva with reasonable recovery (˜70-80%) when samples are diluted 1:2 with the zero standard/blank but that saliva did not work with Crystal Chem assay (saliva insulin not detectable; data not shown). In the current study, it was found that recovery of insulin in spike:recovery tests performed on each plate were somewhat variable between participants (ranging from 40-80%). In the case of low recovery the assays were repeated at higher dilutions (up to 1:4) in order to obtain more accurate saliva insulin values. Notably, low recovery did not appear to impact the interpretation of the results that saliva insulin rises after HC meals and tends to track plasma insulin. However, these data appear to indicate that a ratio between saliva and plasma insulin may not be the same between individuals (i.e., saliva insulin may be ⅓ the value of plasma insulin in one person but ⅕ the value of plasma insulin in another). In such instances, the discrepancy could be addressed by a obtaining fasting insulin value for an individual and measuring the incremental increase in insulin following a high-carbohydrate meal, as opposed to using absolute “cut-off” values.

It was further observed that a better spike:recovery was obtained in subjects with elevated waist circumference (ranging between 70-90%). Moreover, it was found possible to overcome any low spike:recovery issue in saliva samples from some healthy young males by offering about a half (0.5) cup of water to subjects with elevated waist circumference 10 min before collecting their saliva samples. This approach resulted in more clear and higher quality (not viscous) saliva sample and significantly improved spike:recovery for each plate without adversely affecting salivary insulin content in these subjects.

### Results

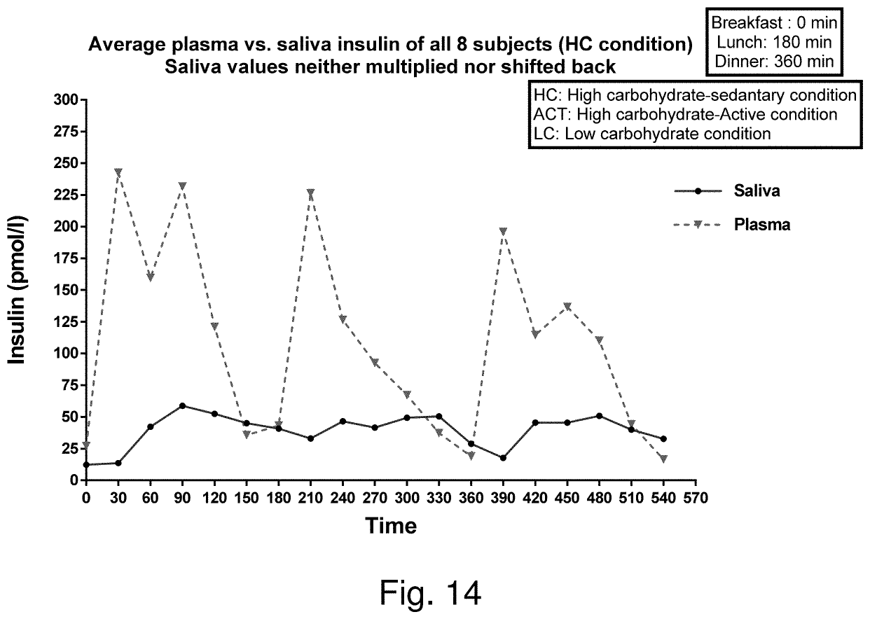

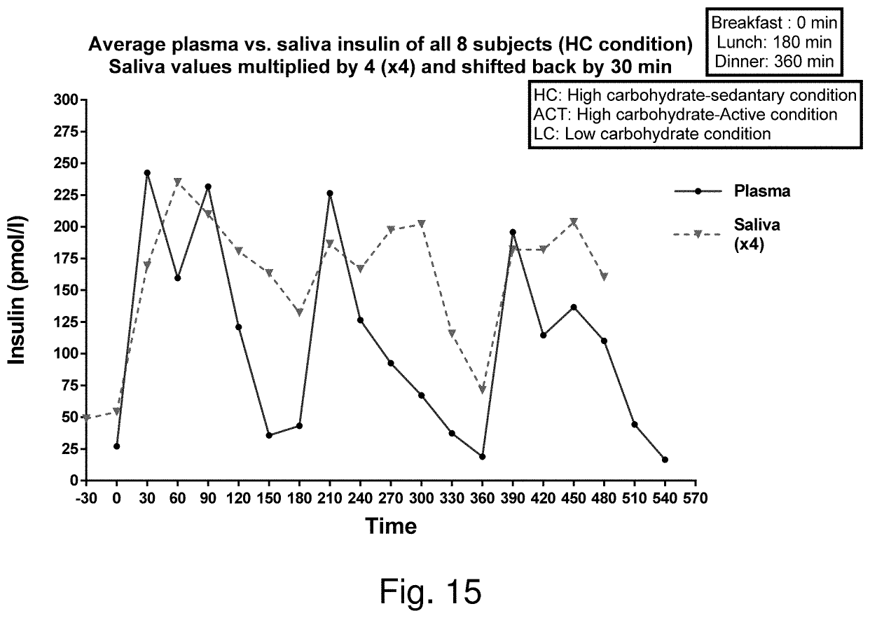

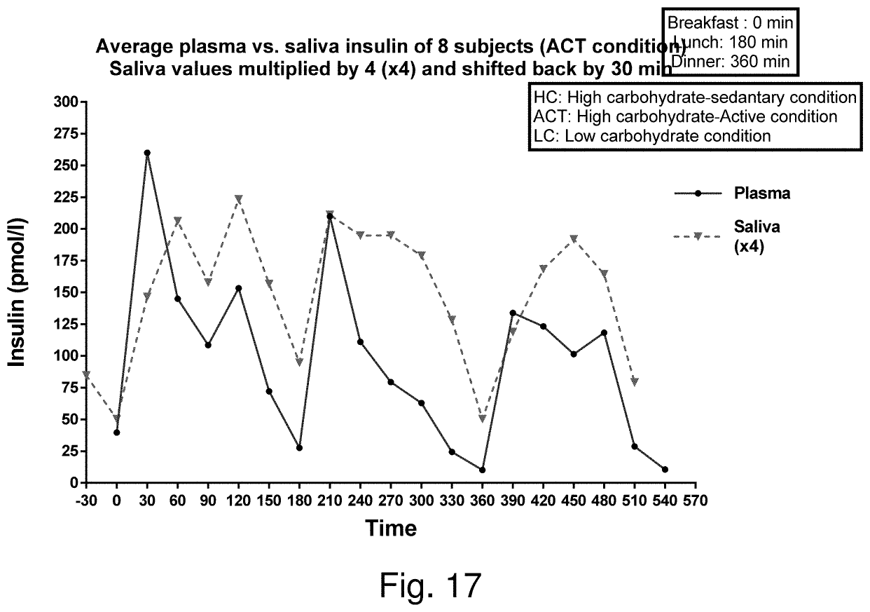

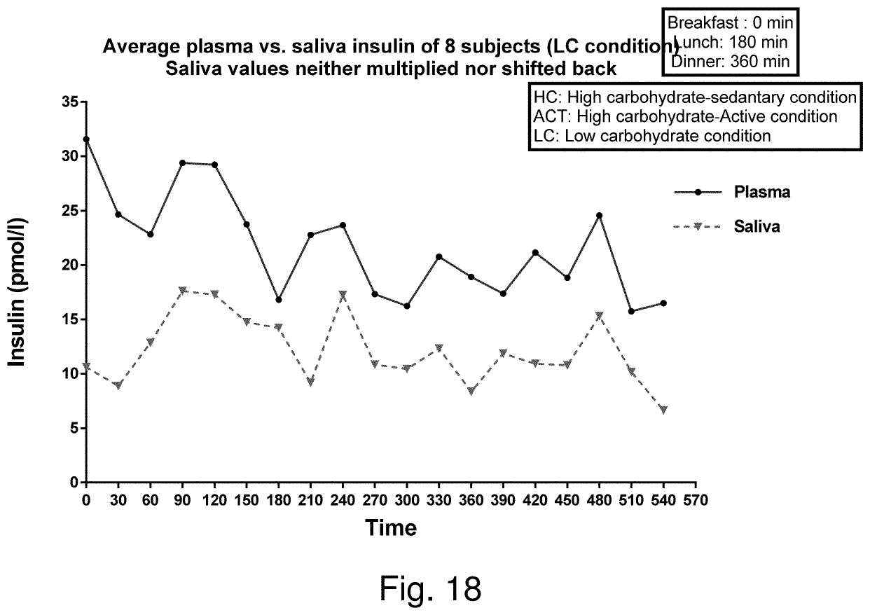

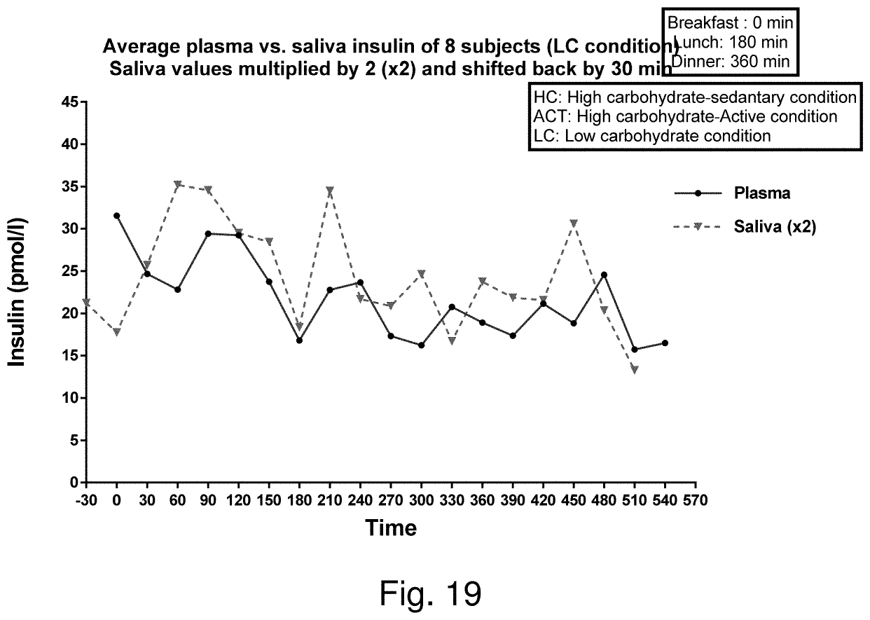

Data were analyzed to produce a saliva and plasma insulin curve/profile for each participant in each condition and also the mean of all participants across all conditions. The curves/profiles representing the mean of all 8 healthy normal weight male participants are shown in FIGS. 14-19 and numerically presented in Table 3.

TABLE 3
Average HC, LC and ACT plasma and saliva insulin (pM)

| Time | Average HC | Average ACT | Average LC | Average HC | Average ACT | Average LC |
|---|---|---|---|---|---|---|
| (min) | saliva (pM) | saliva (pM) | saliva (pM) | Plasma (pM) | Plasma (pM) | Plasma (pM) |
| 0 | 12.22 | 21.15 | 10.61 | 27.18 | 39.76 | 31.57 |
| 30 | 13.57 | 12.58 | 8.87 | 242.57 | 260.04 | 24.65 |
| 60 | 42.31 | 36.60 | 12.86 | 159.53 | 145.01 | 22.81 |
| 90 | 58.79 | 51.57 | 17.60 | 231.69 | 108.57 | 29.40 |
| 120 | 52.47 | 39.43 | 17.28 | 120.93 | 153.28 | 29.22 |
| 150 | 45.17 | 55.84 | 14.75 | 35.70 | 72.14 | 23.73 |
| 180 | 40.85 | 39.17 | 14.21 | 43.19 | 27.65 | 16.81 |
| 210 | 33.08 | 23.69 | 9.18 | 226.43 | 209.99 | 22.76 |
| 240 | 46.58 | 52.79 | 17.24 | 126.53 | 111.02 | 23.66 |
| 270 | 41.63 | 48.69 | 10.85 | 92.57 | 79.44 | 17.32 |
| 300 | 49.34 | 48.70 | 10.43 | 67.25 | 62.93 | 16.23 |
| 330 | 50.51 | 44.74 | 12.32 | 37.31 | 24.46 | 20.77 |
| 360 | 28.91 | 32.04 | 8.36 | 18.94 | 10.07 | 18.91 |
| 390 | 17.81 | 12.59 | 11.87 | 195.75 | 133.88 | 17.36 |
| 420 | 45.50 | 29.72 | 10.93 | 114.52 | 123.26 | 21.14 |
| 450 | 45.47 | 42.08 | 10.78 | 136.60 | 101.36 | 18.83 |
| 480 | 50.91 | 47.94 | 15.30 | 110.15 | 118.24 | 24.56 |
| 510 | 40.06 | 41.06 | 10.17 | 44.35 | 28.79 | 15.74 |
| 540 | 32.76 | 19.80 | 6.64 | 16.51 | 10.55 | 16.49 |

The individual data for each of the 8 healthy normal weight male participants is shown in Tables 4-11. As above, saliva and plasma insulin curves/profiles for each healthy normal weight male participant in each condition were prepared (data not shown).

TABLE 4
HC, ACT and LC data for participant #1 (SI = saliva, PI = plasma)

| Time | HC 1 SI | ACT 1 SI | LC 1 SI | HC 1 PI | ACT 1 PI | LC 1 PI |
|---|---|---|---|---|---|---|
| (min) | (pM) | (pM) | (pM) | (pM) | (pM) | (pM) |
| 0 | 12.30 | 9.90 | 1.50 | 22.50 | 39.06 | 30.66 |
| 30 | 16.14 | 9.54 | 7.44 | 229.26 | 174.78 | 29.40 |
| 60 | 34.74 | 22.92 | 15.00 | 150.60 | 128.76 | 37.08 |
| 90 | 24.48 | 22.74 | 19.02 | 214.02 | 75.72 | 27.06 |
| 120 | 28.86 | 20.40 | 29.40 | 93.84 | 148.62 | 22.92 |
| 150 | 38.10 | 34.26 | 12.30 | 77.22 | 130.74 | 44.64 |
| 180 | 26.94 | 28.62 | 11.04 | 65.46 | 34.44 | 18.06 |
| 210 | 35.52 | 26.76 | 9.24 | 149.94 | 154.80 | 21.18 |
| 240 | 25.92 | 38.76 | 13.68 | 150.90 | 86.16 | 11.40 |
| 270 | 49.50 | 39.72 | 6.78 | 96.72 | 168.84 | 17.76 |
| 300 | 29.82 | 65.22 | 7.26 | 89.82 | 21.60 | 28.08 |
| 330 | 26.64 | 30.48 | 18.84 | 45.60 | 86.88 | 24.66 |
| 360 | 19.50 | 38.82 | 8.10 | 53.28 | 14.58 | 33.42 |
| 390 | 14.52 | 18.36 | 10.08 | 179.16 | 169.74 | 31.74 |
| 420 | 35.40 | 17.10 | 10.14 | 248.52 | 103.14 | 14.22 |
| 450 | 58.50 | 24.48 | 13.14 | 83.22 | 30.24 | 17.04 |
| 480 | 38.40 | 29.52 | 7.38 | 176.64 | 128.04 | 13.86 |
| 510 | 31.38 | 37.86 | 6.54 | 22.92 | 42.36 | 16.32 |
| 540 | 13.68 | 19.98 | 12.48 | 35.70 | 6.00 | 24.96 |

TABLE 5
HC, ACT and LC data for participant #2 (SI = saliva, PI = plasma)

| | HC 2 SI | ACT 2 SI | LC 2 SI | HC 2 PI | ACT 2 PI | LC 2 PI |
|---|---|---|---|---|---|---|
| Time | (pM) | (pM) | (pM) | (pM) | (pM) | (pM) |
| 0 | 14.58 | 33.84 | 15.12 | 30.78 | 42.00 | 10.98 |
| 30 | 9.18 | 19.02 | | 242.04 | 272.16 | 39.12 |
| 60 | 43.32 | 46.74 | 15.12 | 79.20 | 130.98 | 19.50 |
| 90 | 43.32 | 54.84 | 4.26 | 70.32 | 87.54 | 12.48 |
| 120 | 24.30 | 37.32 | 6.12 | 143.46 | 243.00 | 34.08 |
| 150 | 32.94 | 34.50 | 16.02 | 10.56 | 108.90 | 3.84 |
| 180 | 31.56 | 26.28 | 11.04 | 9.24 | 29.10 | 10.32 |
| 210 | 37.44 | 44.04 | 3.90 | 37.86 | 290.70 | 11.76 |
| 240 | 57.60 | 74.04 | 30.60 | 123.24 | 95.34 | 19.80 |
| 270 | 29.58 | 40.32 | 8.10 | 53.76 | 74.16 | 23.94 |
| 300 | 47.28 | 39.12 | 16.50 | 76.50 | 107.10 | 11.40 |
| 330 | 51.60 | 51.24 | 7.44 | 44.46 | 20.16 | 11.76 |
| 360 | 26.16 | 18.30 | 12.12 | 9.24 | 13.20 | 27.06 |
| 390 | 20.64 | 28.56 | 18.42 | 109.68 | 139.38 | 16.32 |
| 420 | 55.56 | 34.80 | 22.80 | 80.70 | 187.74 | 17.40 |
| 450 | 19.62 | 66.78 | 22.62 | 58.14 | 202.44 | 13.14 |
| 480 | 40.98 | 53.58 | 26.40 | 41.16 | 119.70 | 27.36 |
| 510 | 25.56 | 47.28 | 7.32 | 56.16 | 35.40 | 28.08 |
| 540 | 27.12 | 30.54 | 4.56 | 20.16 | 21.00 | 18.06 |

TABLE 6
HC, ACT and LC data for participant #3 (SI = saliva, PI = plasma)

| | HC 3 SI | ACT 3 SI | LC 3 SI | HC 3 PI | ACT 3 PI | LC 3 PI |
|---|---|---|---|---|---|---|
| Time | (pM) | (pM) | (pM) | (pM) | (pM) | (pM) |
| 0 | 34.56 | 50.52 | 26.94 | 42.90 | 55.08 | 36.66 |
| 30 | 28.68 | 29.40 | 29.52 | 331.98 | 369.24 | 30.72 |
| 60 | 133.56 | 112.98 | 49.56 | 377.40 | 225.78 | 28.56 |
| 90 | 183.90 | 199.20 | 80.58 | 431.40 | 66.18 | 74.70 |
| 120 | 231.84 | 152.28 | 67.08 | 219.78 | 360.78 | 32.34 |
| 150 | 193.80 | 221.76 | 33.00 | 46.68 | 38.04 | 37.74 |
| 180 | 171.60 | 153.60 | 39.72 | 91.86 | 73.86 | 36.66 |
| 210 | 104.88 | 58.50 | 33.18 | 436.68 | 330.72 | 38.76 |
| 240 | 188.40 | 140.40 | 25.56 | 193.86 | 227.40 | 21.24 |
| 270 | 165.24 | 216.24 | 41.04 | 190.62 | 52.74 | 36.12 |
| 300 | 237.78 | 188.82 | 25.98 | 86.34 | 189.06 | 19.50 |
| 330 | 207.00 | 165.78 | 49.26 | 58.08 | 27.78 | 42.96 |
| 360 | 117.66 | 153.60 | 22.74 | 17.88 | 13.80 | 22.92 |
| 390 | 28.80 | 11.40 | 39.12 | 375.18 | 281.10 | 33.42 |
| 420 | 137.34 | 119.40 | 34.38 | 207.66 | 168.12 | 20.04 |
| 450 | 202.92 | 172.38 | 37.80 | 322.02 | 58.74 | 25.20 |
| 480 | 231.30 | 175.08 | 44.16 | 96.66 | 156.54 | 24.60 |
| 510 | 141.48 | 167.22 | 42.36 | 32.40 | 14.52 | 20.04 |
| 540 | 136.14 | 58.38 | 23.04 | 18.66 | 6.18 | 15.96 |

TABLE 7
HC, ACT and LC data for participant #4 (SI = saliva, PI = plasma)

| | HC 4 SI | ACT 4 SI | LC 4 SI | HC 4 PI | ACT 4 PI | LC 4 PI |
|---|---|---|---|---|---|---|
| Time | (pM) | (pM) | (pM) | (pM) | (pM) | (pM) |
| 0 | 6.36 | 15.90 | 16.26 | 21.66 | 36.72 | 41.40 |
| 30 | 11.10 | 4.14 | 11.28 | 212.34 | 454.68 | 32.34 |
| 60 | 36.18 | 27.72 | 10.20 | 76.38 | 184.74 | 20.04 |
| 90 | 27.00 | 28.62 | 6.48 | 102.06 | 206.04 | 17.16 |
| 120 | 18.54 | 32.64 | 11.52 | 30.12 | 37.74 | 35.58 |
| 150 | 10.44 | 30.18 | 30.84 | 11.58 | 32.94 | 22.38 |
| 180 | 12.18 | 30.36 | 27.54 | 9.72 | 12.66 | 8.76 |
| 210 | 13.62 | 7.56 | 15.48 | 294.54 | 220.32 | 5.64 |
| 240 | 20.10 | 41.16 | 47.52 | 89.70 | 79.80 | 24.60 |
| 270 | 24.12 | 27.84 | 16.44 | 22.74 | 46.92 | 1.86 |
| 300 | 20.70 | 21.48 | 11.88 | 17.04 | 26.28 | 1.20 |
| 330 | 18.60 | 13.86 | 5.76 | 19.56 | 9.00 | 10.56 |
| 360 | 8.64 | 6.84 | 1.32 | 12.30 | 7.14 | 9.96 |
| 390 | 10.38 | 4.26 | 15.42 | 202.20 | 87.24 | 9.96 |
| 420 | 53.16 | 16.86 | 6.00 | 22.38 | 90.60 | 10.56 |
| 450 | 18.78 | 14.58 | 1.44 | 71.04 | 97.56 | 11.82 |
| 480 | 22.80 | 21.12 | 23.82 | 23.46 | 51.90 | 35.58 |
| 510 | 39.30 | 15.60 | 13.56 | 7.14 | 9.36 | 11.22 |
| 540 | 9.96 | 1.50 | 7.92 | 1.50 | 9.36 | 11.82 |

TABLE 8
HC, ACT and LC data for participant #5 (SI = saliva, PI = plasma)

| | HC 5 SI | ACT 5 SI | LC 5 SI | HC 5 PI | ACT 5 PI | LC 5 PI |
|---|---|---|---|---|---|---|
| Time | (pM) | (pM) | (pM) | (pM) | (pM) | (pM) |
| 0 | 5.94 | 9.00 | 8.40 | 30.60 | 36.06 | 52.44 |
| 30 | 10.02 | 2.94 | 2.76 | 336.36 | 196.26 | 14.64 |
| 60 | 1.08 | 9.06 | 5.46 | 85.02 | 114.00 | 24.36 |
| 90 | 27.06 | 4.32 | 12.72 | 584.10 | 184.50 | 36.30 |
| 120 | 17.94 | 7.56 | 3.36 | 276.60 | 135.00 | 24.36 |
| 150 | 15.84 | 14.88 | 4.92 | 91.32 | 123.60 | 27.42 |
| 180 | 30.84 | 8.82 | 2.64 | 114.96 | 38.76 | 20.46 |
| 210 | 14.94 | 10.08 | 4.80 | 525.54 | 199.68 | 50.04 |
| 240 | 24.30 | 18.66 | 3.48 | 102.06 | 201.48 | 50.04 |
| 270 | 24.72 | 13.92 | 4.38 | 181.74 | 52.56 | 25.68 |
| 300 | 13.68 | 12.00 | 3.66 | 117.72 | 68.64 | 38.76 |
| 330 | 33.90 | 14.10 | 3.90 | 67.26 | 36.42 | 26.52 |
| 360 | 19.44 | 10.50 | 8.40 | 22.26 | 15.78 | 28.68 |
| 390 | 2.76 | 14.70 | 2.34 | 261.60 | 95.58 | 11.40 |
| 420 | 2.10 | 2.28 | 2.22 | 143.82 | 165.78 | 40.38 |
| 450 | 6.24 | 11.46 | 4.32 | 224.16 | 39.90 | 30.42 |
| 480 | 22.08 | 16.68 | 1.50 | 293.16 | 170.28 | 23.52 |
| 510 | 17.76 | 9.60 | 1.08 | 130.50 | 64.44 | 18.24 |
| 540 | 39.12 | 12.48 | 1.26 | 34.92 | 25.08 | 21.78 |

TABLE 9
HC, ACT and LC data for participant #6 (SI = saliva, PI = plasma)

| | HC 6 SI | ACT 6 SI | LC 6 SI | HC 6 PI | ACT 6 PI | LC 6 PI |
|---|---|---|---|---|---|---|
| Time | (pM) | (pM) | (pM) | (pM) | (pM) | (pM) |
| 0 | 3.90 | 10.98 | 3.66 | 12.54 | 25.20 | 24.84 |
| 30 | 2.64 | 7.38 | 4.20 | 177.54 | 120.36 | 14.16 |
| 60 | 20.04 | 8.70 | 0.66 | 186.78 | 96.24 | 8.22 |
| 90 | 40.56 | 14.40 | 1.74 | 80.28 | 36.60 | 14.64 |
| 120 | 12.60 | 10.98 | 1.08 | 15.12 | 31.74 | 10.02 |
| 150 | 12.18 | 10.20 | 3.36 | 11.22 | 18.48 | 10.98 |
| 180 | 6.42 | 9.60 | 6.18 | 6.90 | 8.22 | 7.74 |
| 210 | 8.46 | 5.70 | 0.60 | 95.52 | 100.74 | 32.52 |
| 240 | 11.94 | 25.38 | 0.66 | 57.66 | 48.48 | 22.20 |
| 270 | 7.44 | 12.24 | 1.92 | 20.58 | 21.00 | 3.48 |
| 300 | 13.02 | 5.64 | 1.98 | 28.50 | 13.80 | 8.64 |
| 330 | 16.92 | 2.40 | 1.08 | 27.66 | 2.94 | 9.60 |
| 360 | 7.62 | 3.60 | 1.92 | 5.16 | 6.48 | 10.02 |
| 390 | 5.88 | 2.64 | 1.14 | 64.50 | 79.92 | 16.86 |
| 420 | 14.28 | 9.12 | 1.62 | 64.86 | 25.62 | 19.08 |
| 450 | 10.26 | 8.16 | 0.36 | 32.94 | 36.60 | 14.64 |
| 480 | 9.72 | 12.66 | 3.00 | 17.22 | 30.54 | 26.10 |
| 510 | 27.48 | 4.56 | 1.68 | 14.70 | 6.48 | 14.16 |
| 540 | 4.68 | 0.06 | 0.60 | 2.52 | 6.06 | 10.02 |

TABLE 10
HC, ACT and LC data for participant #7 (SI = saliva, PI = plasma)

| | HC 7 SI | ACT 7 SI | LC 7 SI | HC 7 PI | ACT 7 PI | LC 7 PI |
|---|---|---|---|---|---|---|
| Time | (pM) | (pM) | (pM) | (pM) | (pM) | (pM) |
| 0 | 13.86 | 26.88 | 7.44 | 25.86 | 36.00 | 47.46 |
| 30 | 13.86 | 17.04 | 2.76 | 372.48 | 352.80 | 22.86 |
| 60 | 48.00 | 32.28 | 4.92 | 250.14 | 172.20 | 27.06 |
| 90 | 91.92 | 48.54 | 2.88 | 315.42 | 199.02 | 27.54 |
| 120 | 68.82 | 31.08 | 4.26 | 183.12 | 261.78 | 69.06 |
| 150 | 31.86 | 77.28 | 5.16 | 31.62 | 109.32 | 41.88 |
| 180 | 16.62 | 43.74 | 7.14 | 35.58 | 17.64 | 25.98 |
| 210 | 43.98 | 34.20 | 4.38 | 198.18 | 281.04 | 14.10 |
| 240 | 27.48 | 60.30 | 7.62 | 258.18 | 108.60 | 35.10 |
| 270 | 20.10 | 23.76 | 3.66 | 121.56 | 197.04 | 27.06 |
| 300 | 20.28 | 34.02 | 4.32 | 111.36 | 51.60 | 19.62 |
| 330 | 21.36 | 54.78 | 2.76 | 29.88 | 11.52 | 11.28 |
| 360 | 20.88 | 12.00 | 4.86 | 12.48 | 8.64 | 18.00 |
| 390 | 53.52 | 12.60 | 5.10 | 283.50 | 129.24 | 18.00 |
| 420 | 54.36 | 27.78 | 5.22 | 92.70 | 217.20 | 40.92 |
| 450 | 22.68 | 22.68 | 3.12 | 261.30 | 281.76 | 28.08 |
| 480 | 27.24 | 41.52 | 8.16 | 224.58 | 240.96 | 33.60 |
| 510 | 25.32 | 26.58 | 5.22 | 46.62 | 54.00 | 14.64 |
| 540 | 22.68 | 20.10 | 2.52 | 18.12 | 10.08 | 21.78 |

TABLE 11
HC, ACT and LC data for participant #8 (SI = saliva, PI = plasma)

| | HC 8 SI | ACT 8 SI | LC 8 SI | HC 8 PI | ACT 8 PI | LC 8 PI |
|---|---|---|---|---|---|---|
| Time | (pM) | (pM) | (pM) | (pM) | (pM) | (pM) |
| 0 | 6.24 | 12.18 | 5.58 | 30.60 | 47.94 | 8.10 |
| 30 | 16.92 | 11.16 | 4.14 | 38.52 | 140.04 | 13.98 |
| 60 | 21.54 | 32.40 | 1.92 | 70.74 | 107.34 | 17.64 |
| 90 | 32.04 | 39.90 | 13.08 | 55.92 | 12.96 | 25.32 |
| 120 | 16.86 | 23.16 | 15.42 | 5.40 | 7.56 | 5.40 |
| 150 | 26.22 | 23.64 | 12.36 | 5.40 | 15.06 | 0.96 |
| 180 | 30.60 | 12.36 | 8.40 | 11.82 | 6.48 | 6.48 |
| 210 | 5.76 | 2.70 | 1.86 | 73.14 | 101.94 | 8.10 |
| 240 | 16.92 | 23.58 | 8.82 | 36.60 | 40.92 | 4.86 |
| 270 | 12.30 | 15.48 | 4.44 | 52.86 | 22.26 | 2.64 |
| 300 | 12.12 | 23.28 | 11.88 | 10.68 | 25.32 | 2.64 |
| 330 | 28.08 | 25.26 | 9.48 | 6.00 | 0.96 | 28.80 |
| 360 | 11.40 | 12.66 | 7.38 | 6.00 | 0.96 | 1.20 |
| 390 | 5.94 | 8.22 | 3.30 | 90.18 | 88.86 | 1.20 |
| 420 | 11.76 | 10.38 | 5.04 | 55.50 | 27.84 | 6.48 |
| 450 | 24.78 | 16.14 | 3.42 | 39.96 | 63.66 | 10.26 |
| 480 | 14.76 | 33.36 | 7.98 | 8.34 | 47.94 | 11.88 |
| 510 | 12.18 | 19.74 | 3.60 | | 3.78 | 3.18 |
| 540 | 8.70 | 15.36 | 0.72 | 0.48 | 0.60 | 7.56 |

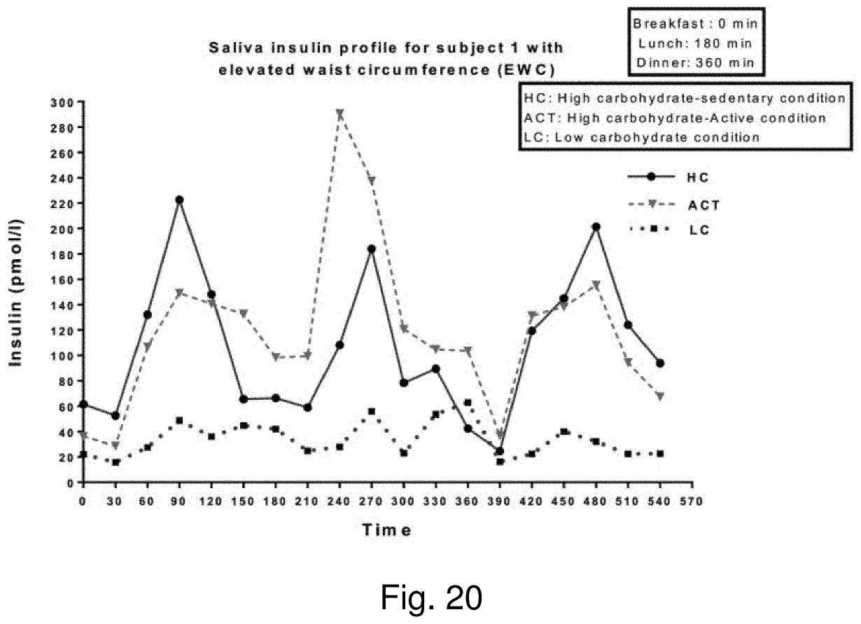

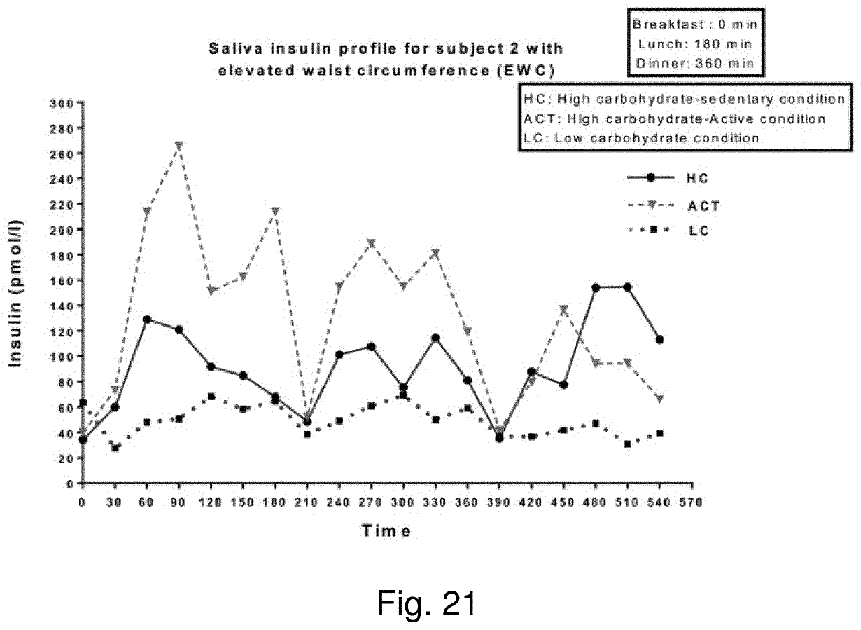

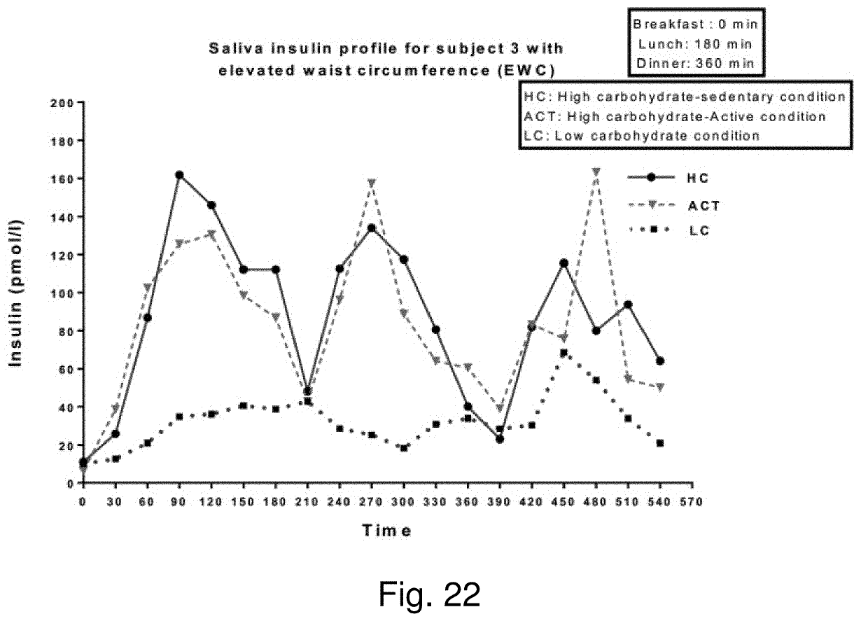

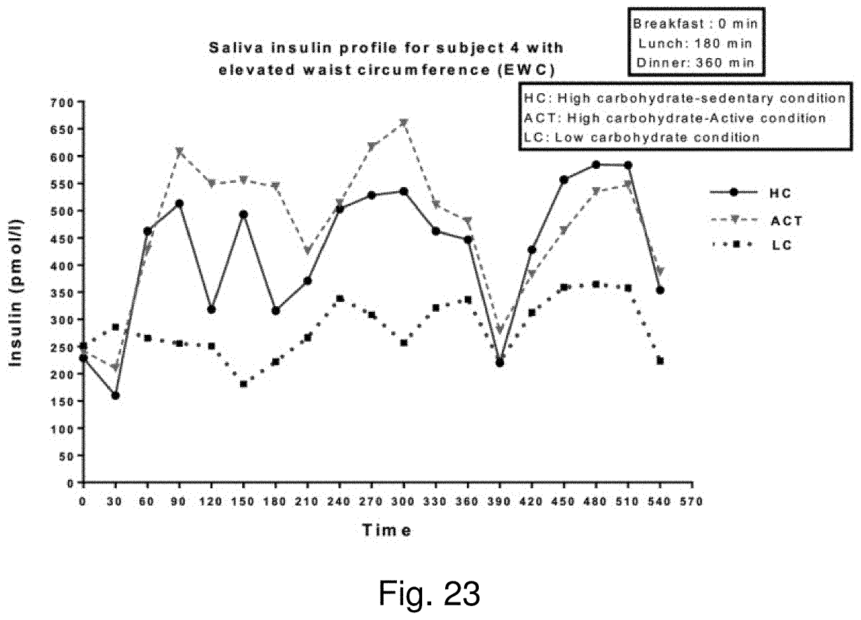

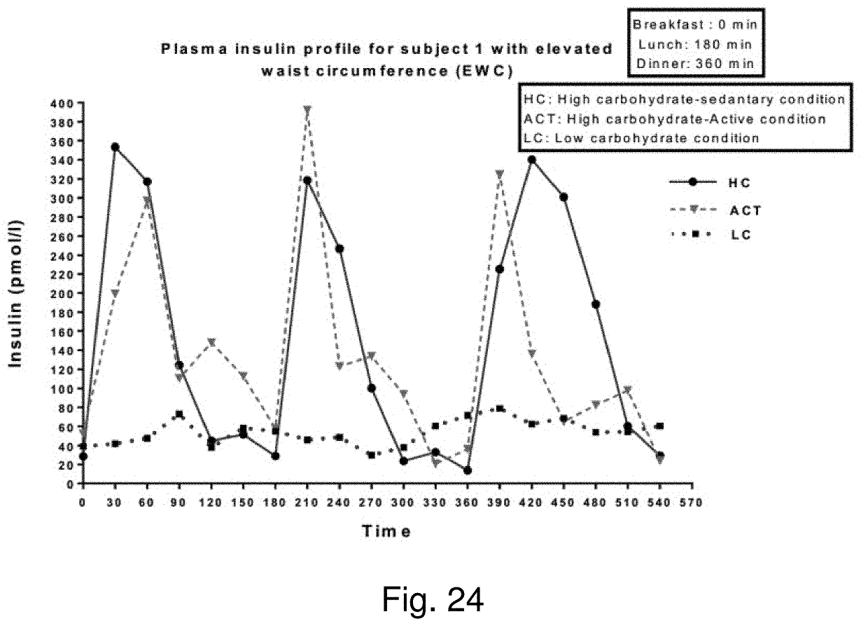

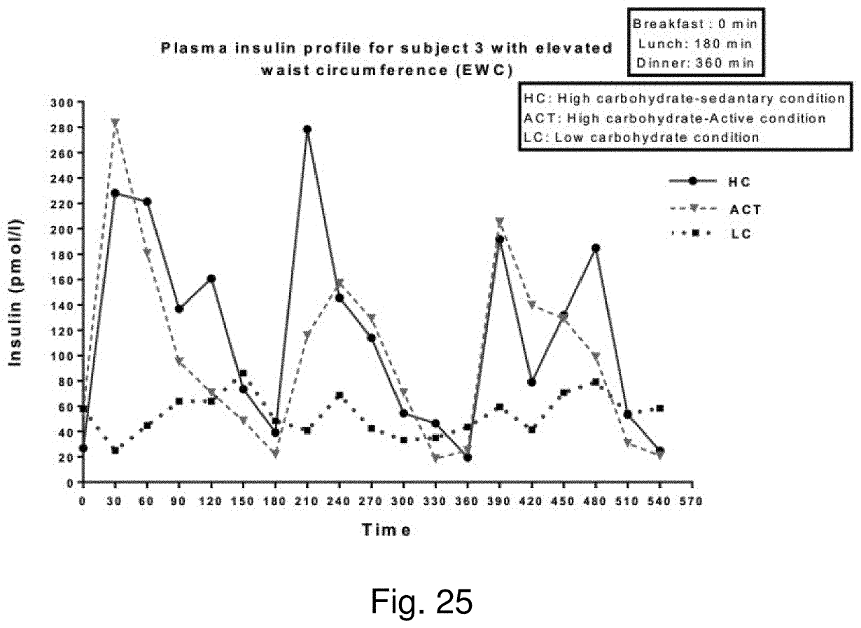

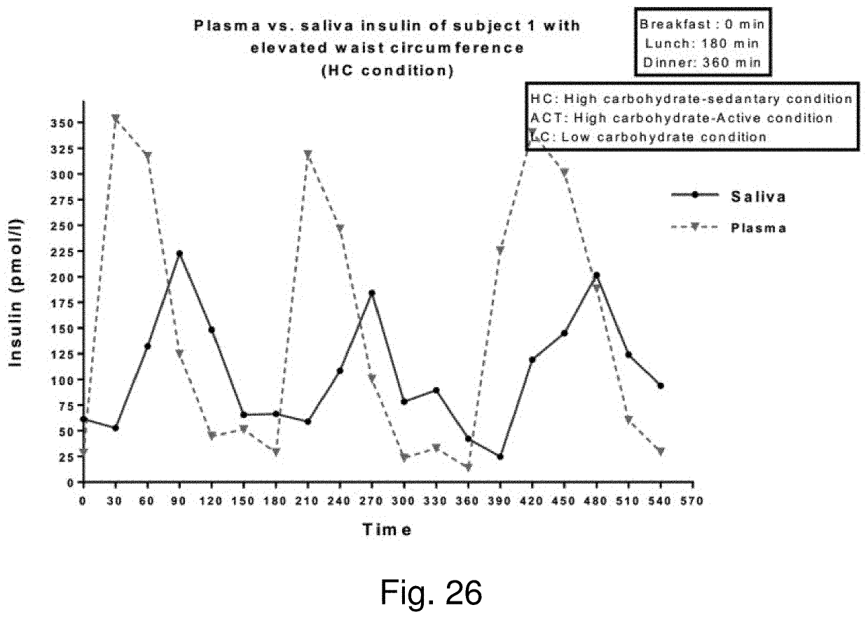

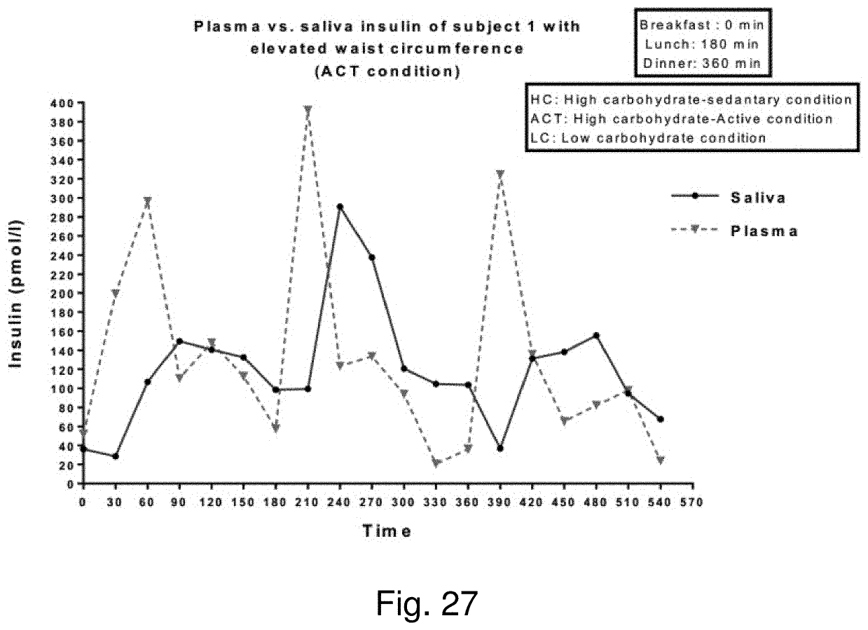

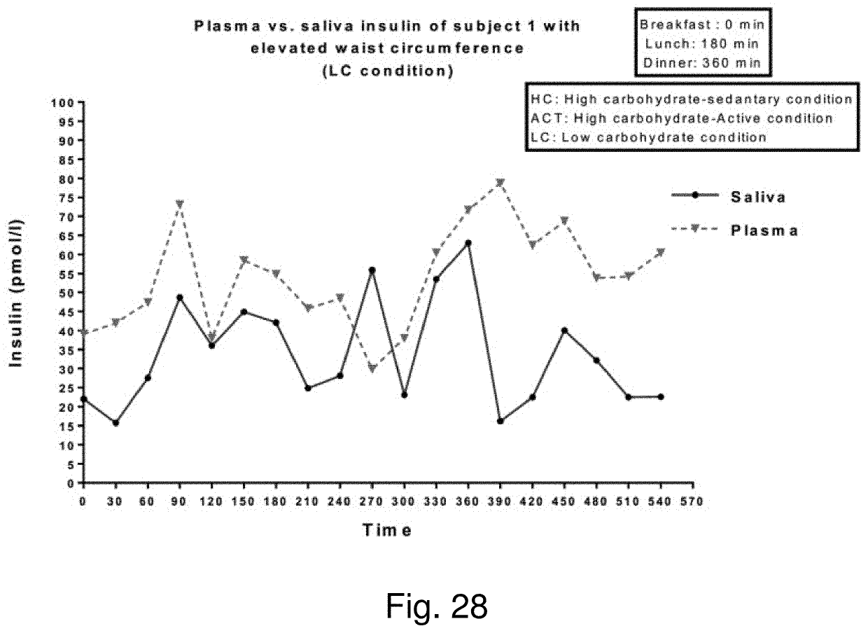

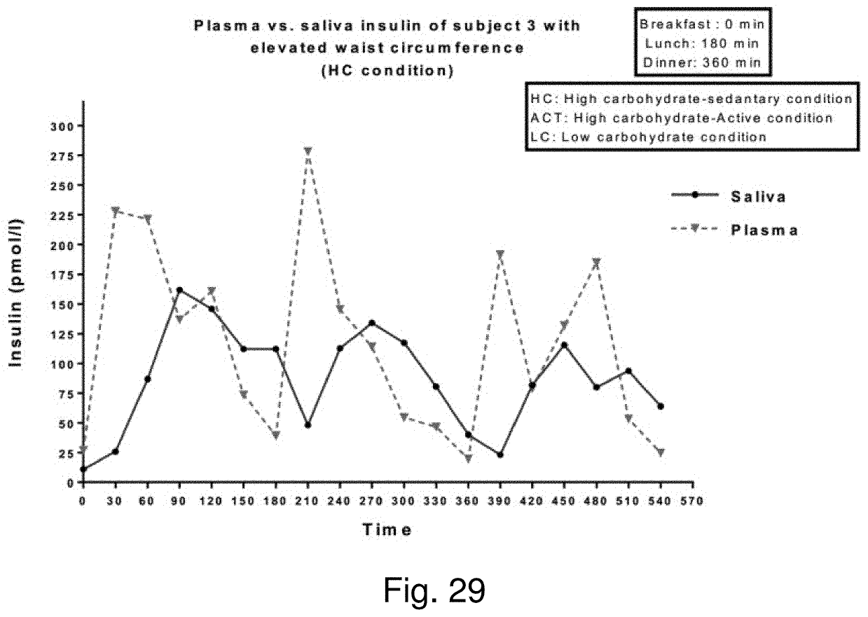

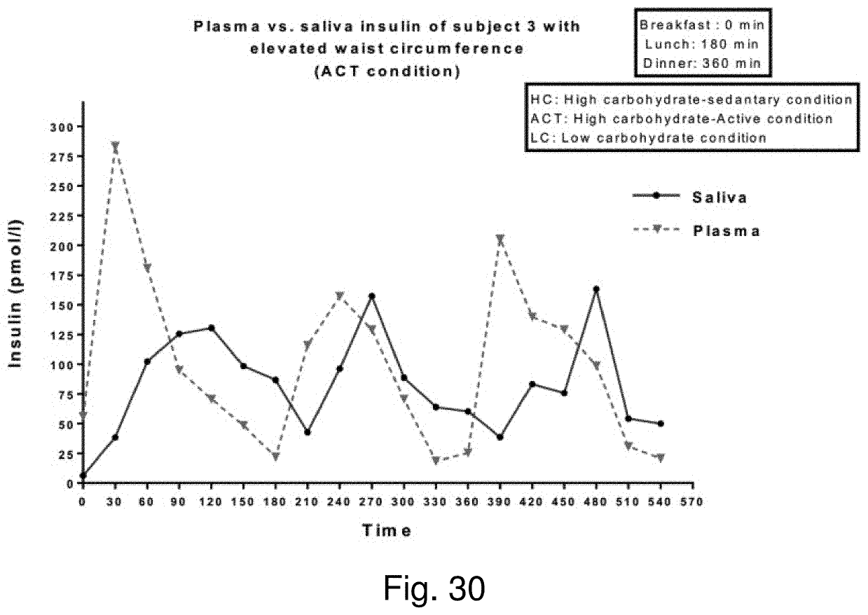

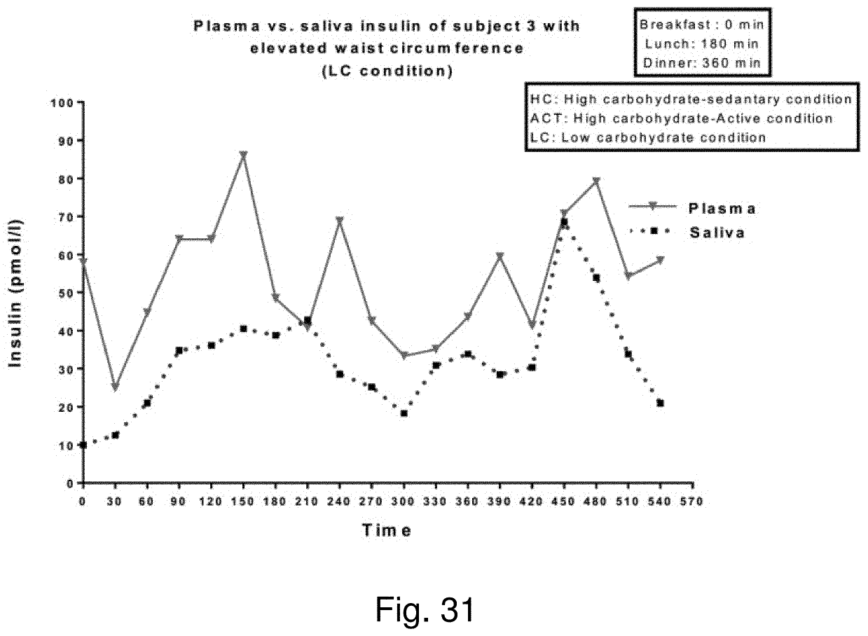

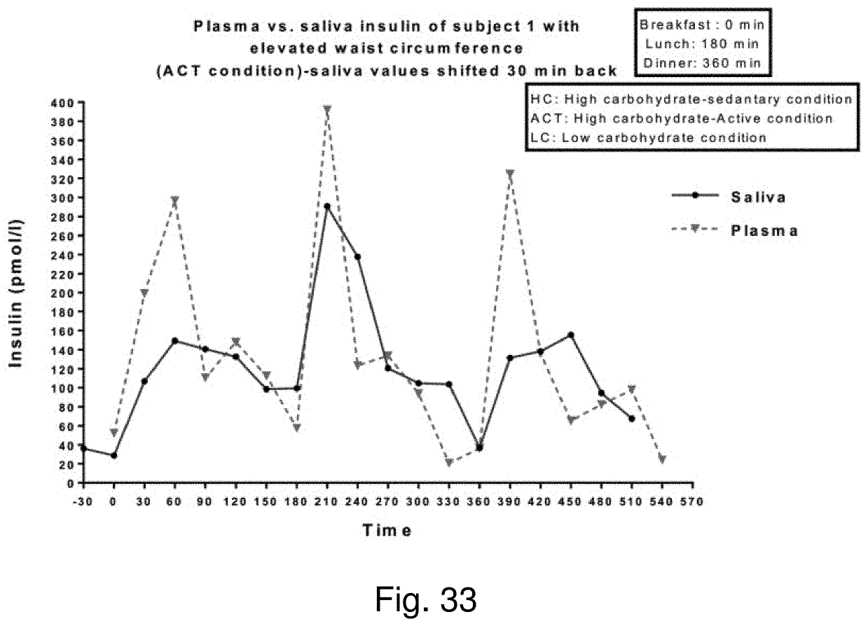

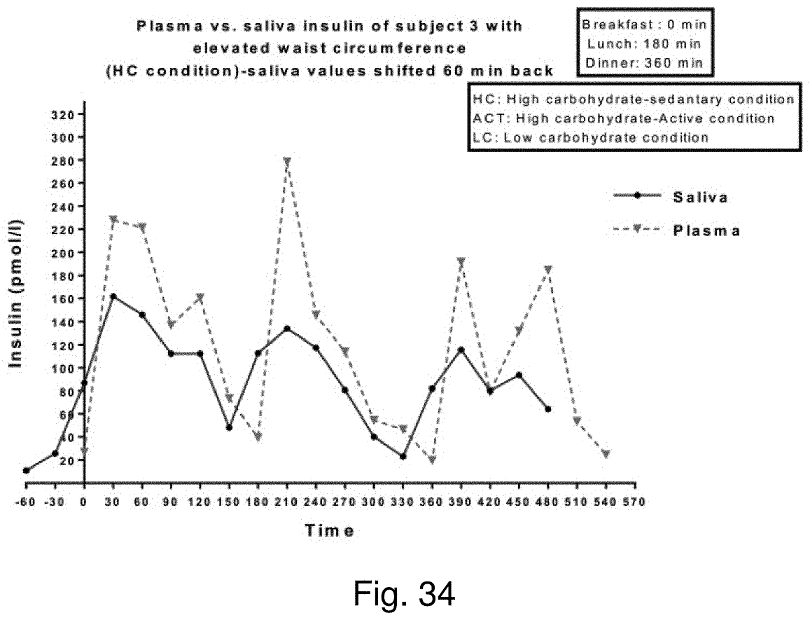

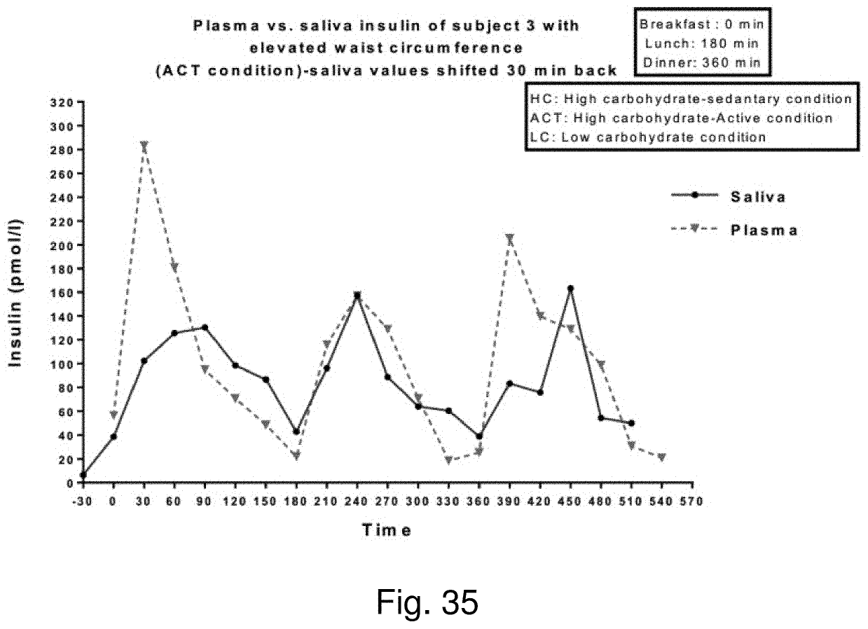

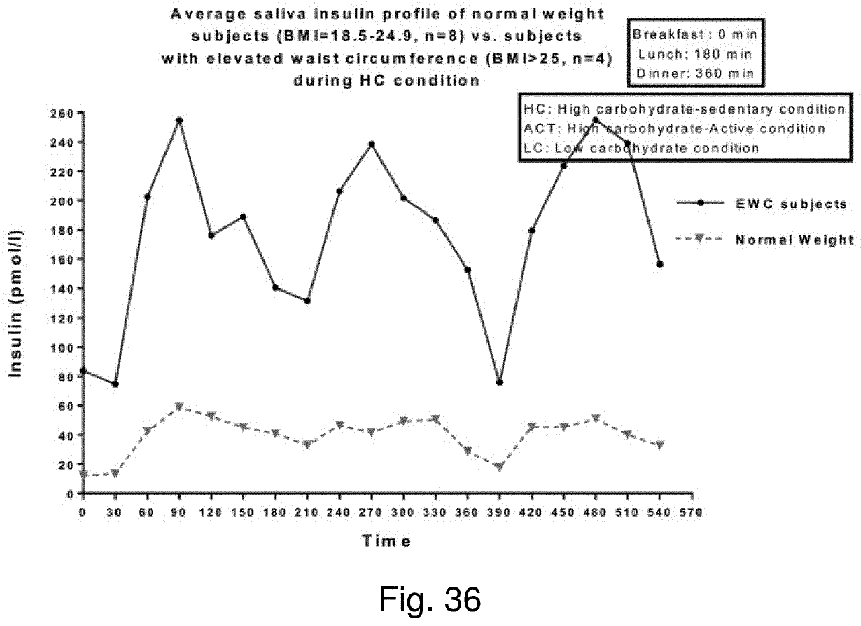

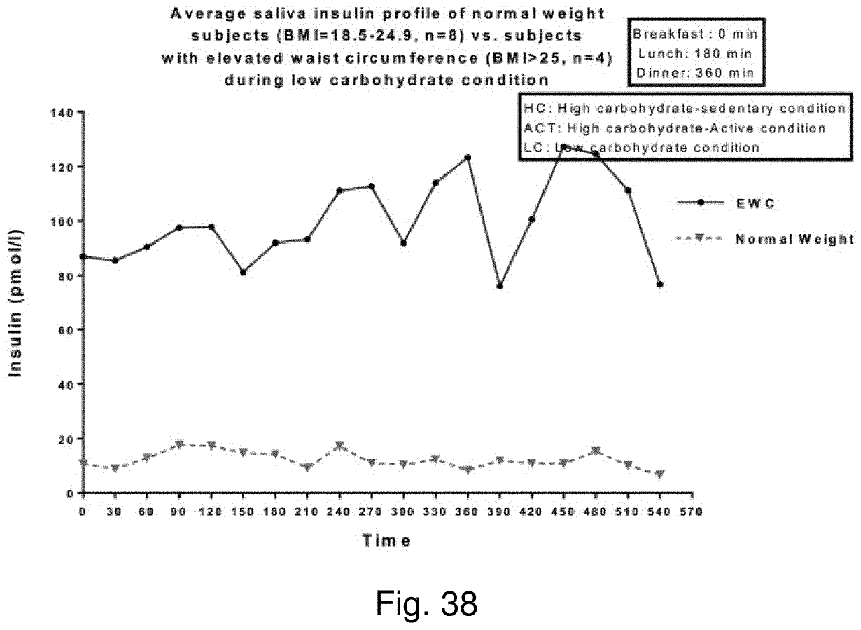

The individual saliva and plasma insulin curves for subjects with elevated waist circumference separated by each condition are shown in FIGS. 20-35 (except plasma insulin values for participant 2 and 4 due to failed insertion of IV Line). The mean of saliva insulin for all healthy normal weight male participants vs. subjects with elevated waist circumference separated by each condition are shown in FIGS. 36-38.

It was observed that for the majority of participants in the HC and ACT conditions, the saliva insulin profile “matched” the plasma insulin if the saliva values were multiplied by about 4 times (×4) and shifted to account for an about 30 minute delay. This means that on average saliva insulin is 25-50% of plasma insulin (on an absolute sense using the ELISA assays) and lags behind plasma by about 30 minutes. In the LC condition, where the insulin spikes were smaller and blunted (as expected), the saliva profile more closely matched the plasma profile when the saliva values were multiplied by 2 times (×2). The lag was not as clear likely because there is very little fluctuation in insulin when low-carb foods are consumed so there is little room for visualizing/detecting low spikes around the baseline.

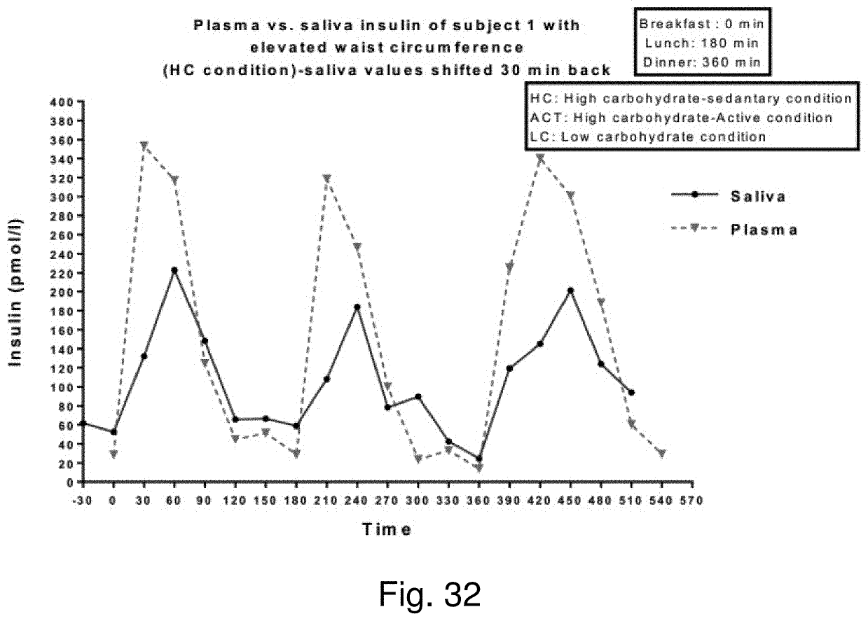

It was observed that there was a higher baseline (fasting) saliva insulin and larger spike in plasma insulin in response to meals in subjects with elevated waist circumference compared to healthy normal weight male subjects. This shows that saliva insulin in the fasted state and post-meals will be higher in participants with elevated waist circumference (i.e., when participants are expected to have some degree of insulin resistance, saliva insulin is higher). It was observed that in participants with elevated waist circumference in HC and ACT conditions, saliva insulin profile “matched” plasma insulin if the saliva was shifted to account for an about 30 minute delay but, interestingly, no multiplying for saliva values was needed for these participants (see FIGS. 32-35). This was possibly related to the improved clarity of saliva samples with water taken 10 min before saliva collection. Also, in these participants saliva insulin profile “matched” plasma insulin in the LC condition without the need for shifting or multiplying saliva values. Thus, consistent with the findings in males with normal healthy weight, in subjects with elevated waist circumference in LC condition saliva insulin “matched” plasma insulin without any lag.

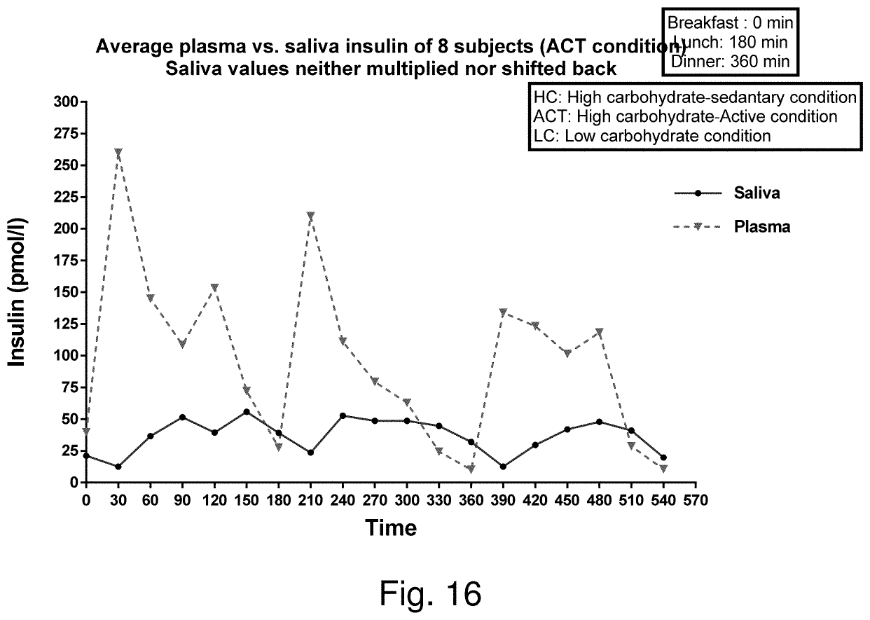

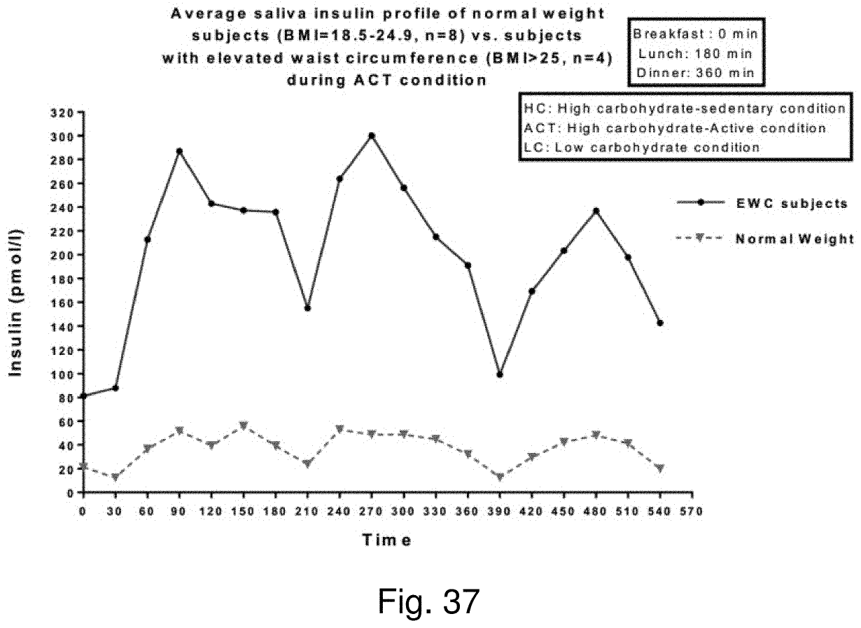

As shown in FIGS. 14-19 and Table 3 for group averages and Tables 4-11 for individual data for males with normal healthy weight, and FIGS. 20-38 for subjects with elevated waist circumference, it was found that after the HC meals saliva insulin returns close to baseline levels after about 180 minutes (3 hours) following each meal. Similar to Example 1, the peak saliva insulin after breakfast (first meal of the day) occurs at about 60-90 minutes in the HC condition, although when data were averaged (FIGS. 14-19 and Table 3 for males with healthy normal weight and FIGS. 36-37 for subject with elevated waist circumference) there appeared to be a biphasic/two-peak response overall. After lunch and dinner (3 and 6 hours later) the peak response timing appeared to be slightly earlier, occurring on the average at 30 minutes post-lunch and post-dinner (FIGS. 14 and 16), although there was some variability in these responses between individuals (Tables 4-11). Peaks after LC meals are harder to identify likely related to the fact that the spike (in both plasma and insulin) is low.

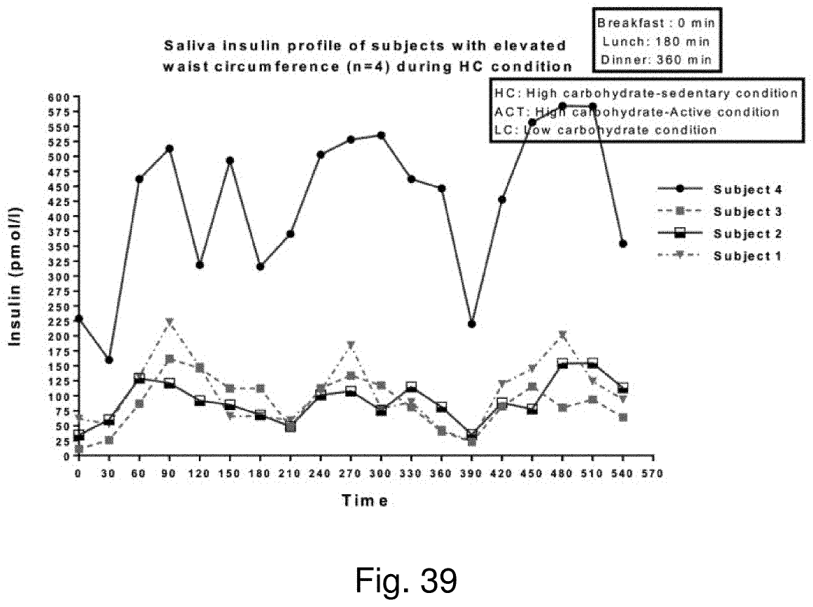

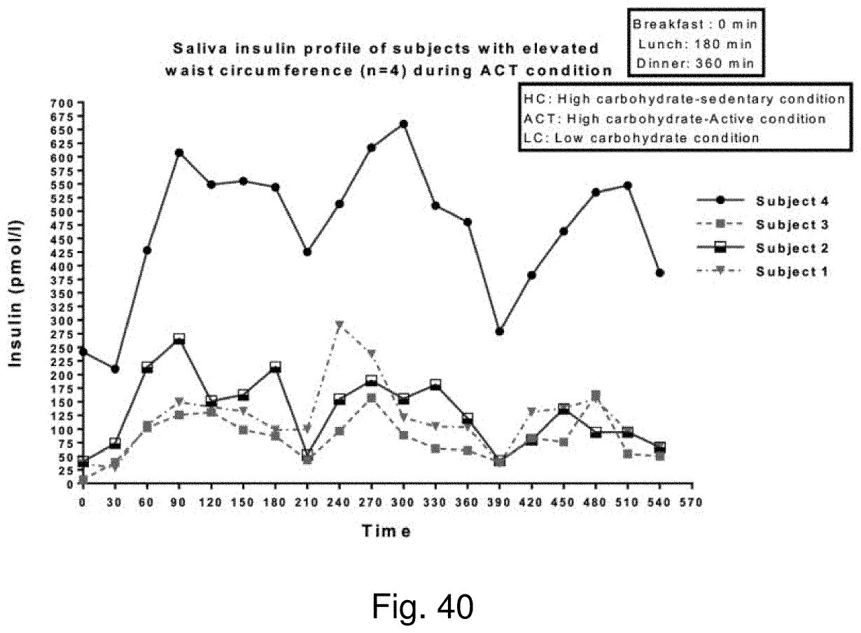

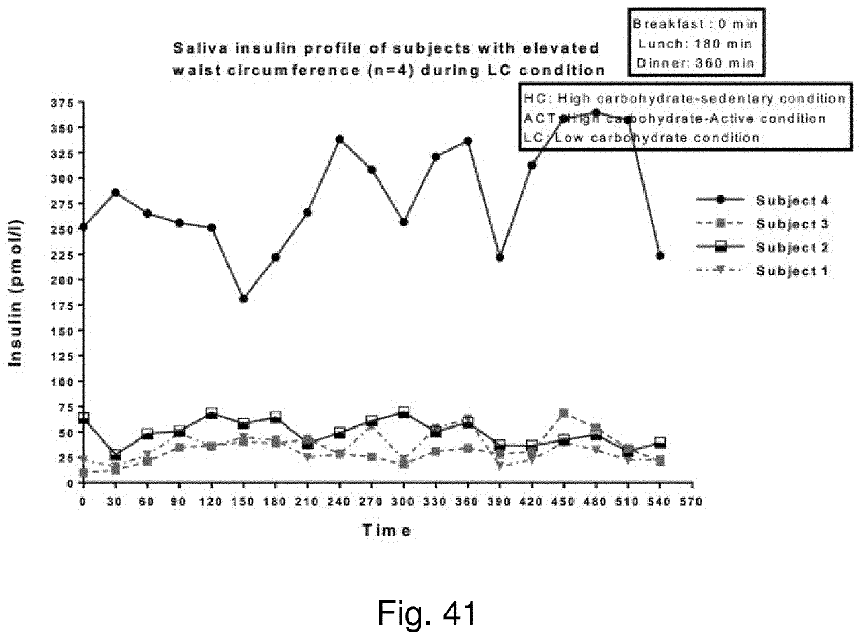

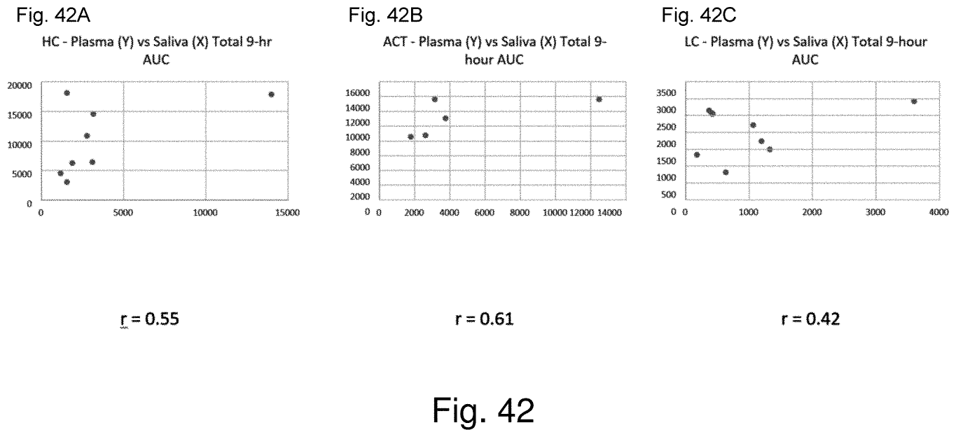

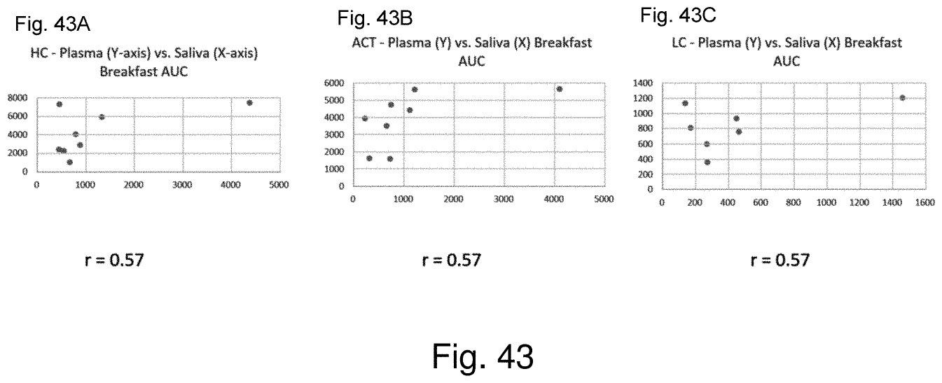

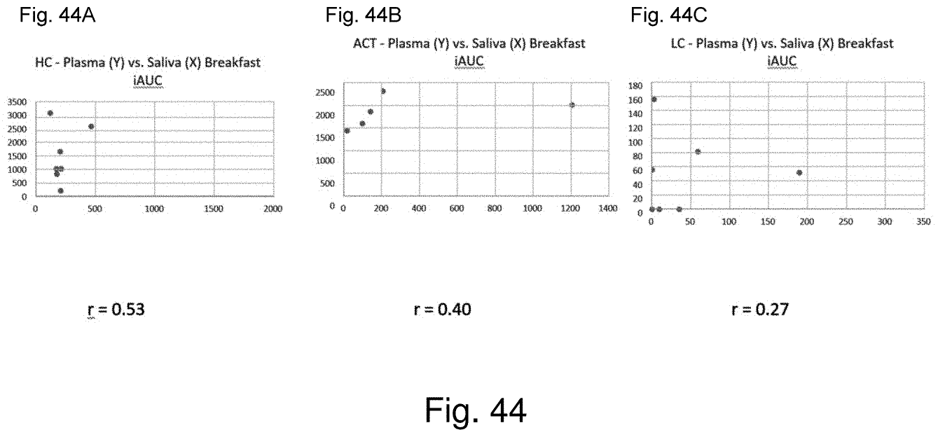

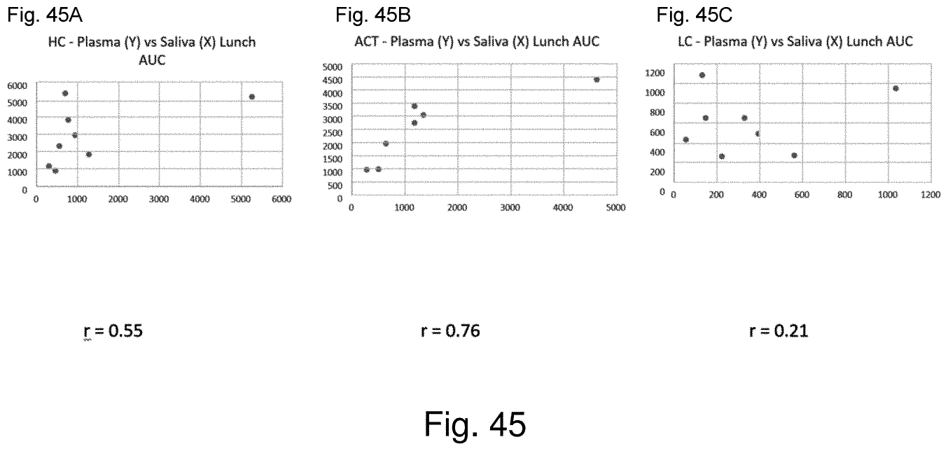

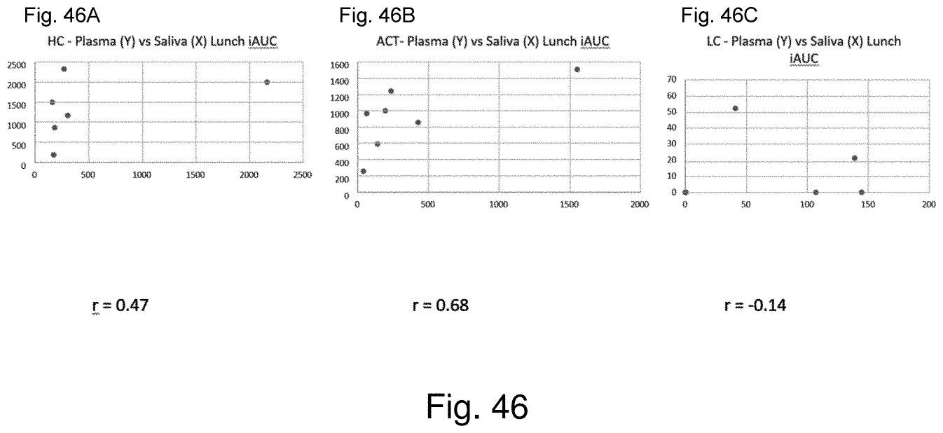

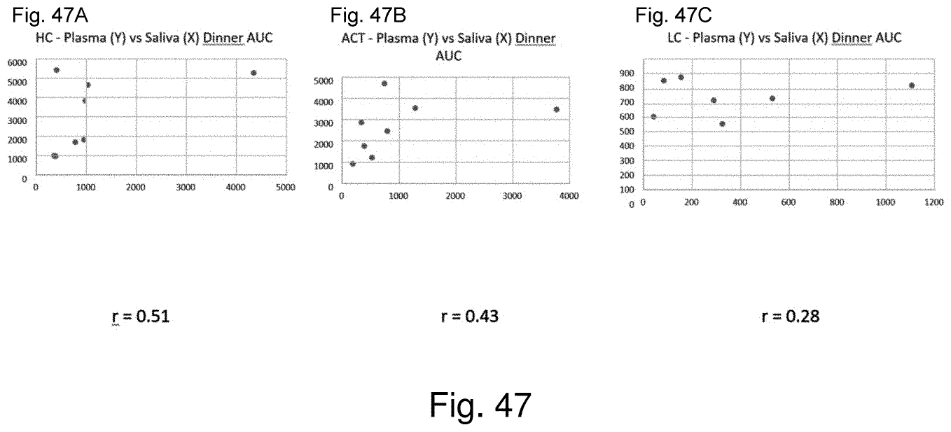

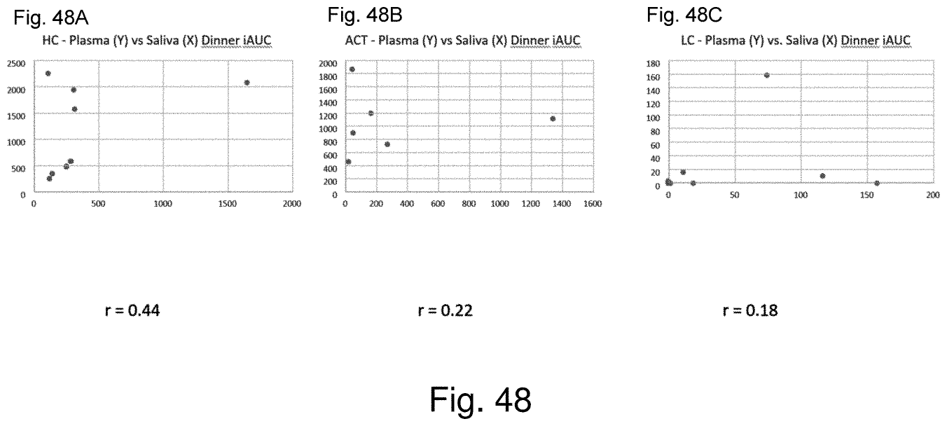

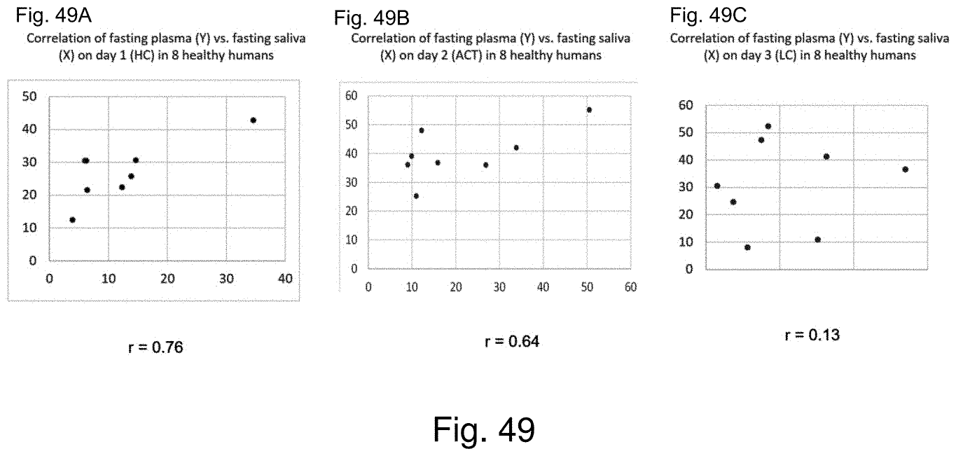

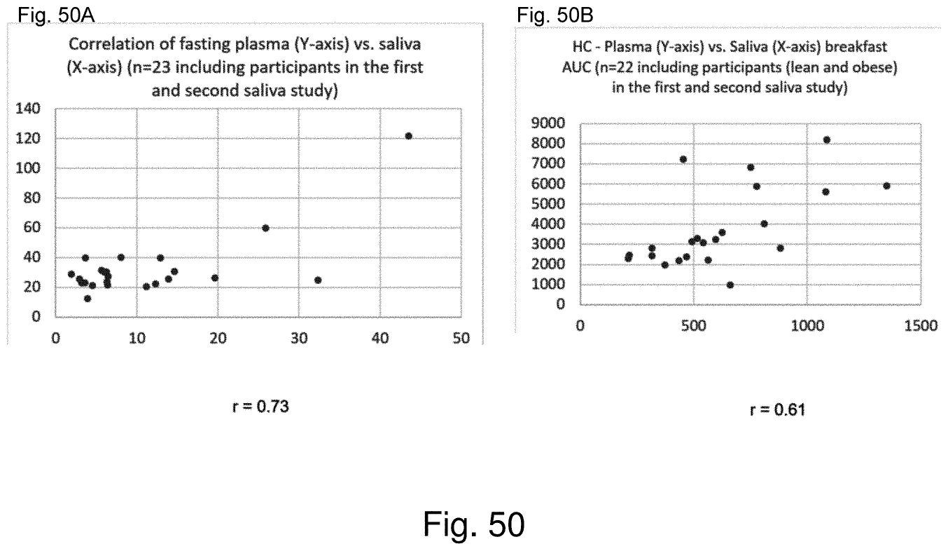

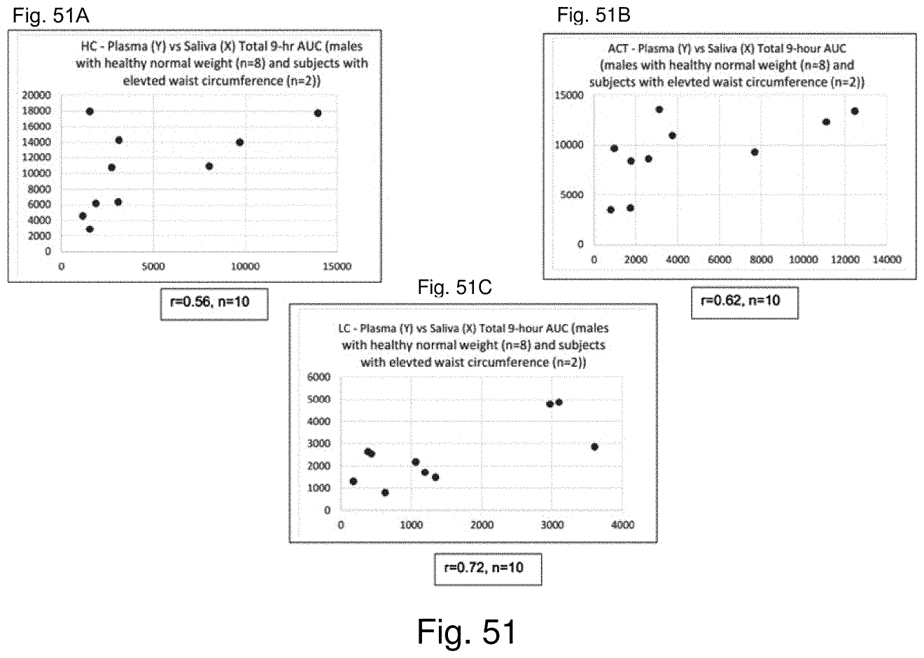

In order to determine whether saliva insulin profiles and responses overall reflect plasma insulin, the area under the curve (AUC) and incremental AUC (above baseline) was calculated for the total 9-hour day and for 3-hour following each meal. Results are shown in FIGS. 39-51. In general, the results show that the saliva insulin response to a high-carbohydrate meal, as reflected by both the AUC or iAUC, matches the plasma response in that participants with low post-meal saliva insulin responses have low post-meal plasma insulin responses. This was true for breakfast, lunch, and dinner in both HC and ACT, with moderate Pearson r-value correlations typically around r=0.5. It can be seen that one young, healthy male had high plasma and saliva insulin response (high AUCs—data point on top right of all figures) indicative of insulin resistance that could be detected by both saliva and plasma responses, in line with the idea that saliva insulin can delineate insulin resistance similar to plasma. It is likely that these correlations would become stronger with inclusion of participants with higher insulin responses in order to provide a greater range of values. For example, it was observed that by taking into account the saliva and plasma total AUC of participants with elevated waist circumference (n=2, given that it was not possible to collect plasma samples from the other 2 participants with elevated waist circumference), the Pearson r-value correlation increased from 0.54 to 0.56 in HC condition, from 0.6 to 0.62 in ACT condition, and from r=0.42 to r=0.72 in LC condition. Also, excluding saliva and plasma AUC of one participant (potential outlier) from the data (n=9) could increase Pearson r-value correlation from 0.56 to 0.8 in HC condition and from r=0.62 to 0.75 in ACT condition.

The overall 9-hour AUC correlation between saliva and plasma insulin was moderate (r=0.56 for HC and r=0.62 for ACT) suggesting that overall “exposure” to insulin throughout the day can be accurately tracked by saliva. This can also be seen in the insulin profile figures, which visually support that saliva insulin accurately tracks plasma insulin when high-carbohydrate meals are consumed (in HC and ACT). Performing exercise does not seem to alter the validity of saliva insulin responses.

Overall, the results of this study show that saliva insulin can be used to reflect plasma insulin responses to separate meals throughout the day. Overall, saliva insulin tends to be lower than the value measured in plasma and excursions after meals tend to lag about 30 minutes behind plasma (except in the LC condition in which no lag was observed in most participants). Also, higher baseline and post-meal saliva insulin was observed in subjects with elevated waist circumference that returned to baseline levels almost 3 hours after each meal. Saliva insulin appears more reflective of plasma insulin after high-carbohydrate meals (when insulin spikes are higher) and the addition of exercise does not appear to alter the relationship between saliva and plasma insulin after consuming high-carbohydrate meals. This indicates that performing activity in real-life would not invalidate saliva insulin measures.

The peak saliva insulin response after a high-carbohydrate meal (at breakfast, lunch, or dinner) occurs at about 60-90 minutes post-meal, with some greater variability at lunch and dinner meals. After breakfast, lunch, and dinner meals, saliva insulin appears to return to basal at approximately 180 minutes, but there is some variability in this response. Saliva insulin can be used to reflect overall high vs. low insulin levels after breakfast, lunch, and dinner meals. There is an overall low saliva insulin spike/response after low-carbohydrate meals, which supports the use of saliva insulin to track/monitor insulin after eating in order to maintain low insulin for weight loss or metabolic health.

### References:

1) Atkinson, F. S.; Foster-Powell, K.; and Brand-Miller, J. C. (2008) “International Tables of Glycemic Index and Glycemic Load Values”, Diab Care, 31:2281-2283.

2) Consultation, W. E. (2008) “Waist Circumference and Waist-Hip Ratio”, Report of a WHO Expert Consultation, World Health Organization: Geneva, Switzerland, pp. 8-11.

3) Corkey, B. E. (2012a) Banting Lecture 2011, Diabetes, 61:4-13.

4) Corkey, B. E. (2012b) “Diabetes: Have we got it all wrong?”, Diabetes Care, 35:2432-2437.

5) Gulli, G.; Ferrannini, E ; Stern, M.; Haffner, S.; and DeFronzo, R. A. (1992) “The metabolic profile of niddm is fully established in glucose-tolerant offspring of two mexican-american niddm parents”, Diabetes, 41:1575-1586.

6) Hu, J.; Wang, T.; Kim, J.; Shannon, C.; and Easley, C. (2012) “Quantification of Femtomolar Protein Levels via Direct Readout with the Electrochemical Proximity Assay, J. Am. Chem. Soc., 134:7066-7072.

7) Hu, J.; Yu, Y.; Brooks, J. C.; Godwin, L. A.; Somasundaram, S.; Torabinejad, F.; Kim, J.; Shannon, C. and Easley, C. J. (2014) “A Reusable Electrochemical Proximity Assay for Highly Selective, Real-Time Protein Quantification in Biological Matrices”, J. Am. Chem. Soc., 136:8467-8474.

8) Kashyap, S. R.; Belfort, R.; Berria, R.; Suraamornkul, S.; Pratipranawatr, T.; Finlayson, J.; Barrentine, A.; Bajaj, M.; Mandarino, L.; and DeFronzo, R. (2004) “Discordant effects of a chronic physiological increase in plasma ffa on insulin signaling in healthy subjects with or without a family history of type 2 diabetes”, Am. J. Physiol. Endocrinol. Metab., 287:E537-E546.

9) Lillioja, S.; Mott, D. M.; Spraul, M.; Ferraro, R.; Foley, J. E.; Ravussin, E.; Knowler, W. C.; Bennett, P. H.; and Bogardus, C. (1993) “Insulin resistance and insulin secretory dysfunction as precursors of non-insulin-dependent diabetes mellitus: Prospective studies of pima indians”, N. Engl. J. Med. 329:1988-1992.

10) Mehran, A. E.; Templeman, N. M.; Brigidi, G. S.; Lim, G. E.; Chu, K. -Y.; Hu, X.; Botezelli, J. D.; Asadi, A.; Hoffman, B. G.; and Kieffer, T. J. (2012) “Hyperinsulinemia drives diet-induced obesity independently of brain insulin production”, Cell Metab., 16:723-737.

11) Myette-Côté, E.; Baba, K.; Brar, R. and Little, J. P. (2017) “Detection of salivary insulin following low versus high carbohydrate meals in humans, Nutrients, 9:1204-1213.

12) Pallotta, J. A.; and Kennedy, P. J. (1968) “Response of plasma insulin and growth hormone to carbohydrate and protein feeding”, Metabolism, 17:901-908.

13) Pratipanawatr, W.; Pratipanawatr, T.; Cusi, K.; Berria, R.; Adams, J. M.; Jenkinson, C. P.; Maezono, K.; DeFronzo, R. A.; and Mandarino, L. J. (2001) “Skeletal muscle insulin resistance in normoglycemic subjects with a strong family history of type 2 diabetes is associated with decreased insulin-stimulated insulin receptor substrate-1 tyrosine phosphorylation”, Diabetes 50:2572-2578.

14) Templeman, N. M.; Skovso, S.; Page, M. M.; Lim, G. E.; Johnson, J. D. (2017) “A causal role for hyperinsulinemia in obesity”, J. Endocrinol. 232:R173-R183.

15) Van Loon, L. J.; Saris, W. H.; Verhagen, H.; and Wagenmakers, A. J. (2000) “Plasma insulin responses after ingestion of different amino acid or protein mixtures with carbohydrate”, Am. J. Clin. Nutr., 72:96-105.

16) Warram, J. H.; Martin, B. C.; Krolewski, A. S.; Soeldner, J. S.; and Kahn, C. R. (1990) “Slow glucose removal rate and hyperinsulinemia precede the development of type ii diabetes in the offspring of diabetic parents”, Ann. Intern. Med., 113:909-915.

1. A method for detecting or predicting a postprandial blood or plasma insulin response to a foodstuff in a subject, said method comprising:
obtaining a first saliva sample from the subject;
obtaining a second saliva sample from the subject following ingestion of a foodstuff;
measuring a first level of insulin in the first saliva sample;
measuring a second level of insulin in the second saliva sample; and
characterizing the subject's blood or plasma insulin level based upon a difference between the first level of insulin and the second level of insulin in saliva, to thereby detect or predict a postprandial blood or plasma insulin response to the foodstuff.

2. (canceled)

3. The method of claim 1, wherein the second saliva sample is taken from the subject at least 30 minutes after ingestion of the foodstuff.

4. The method of claim 1, further comprising: obtaining one or more additional saliva samples from the subject at different times following ingestion of the foodstuff and measuring one or more additional levels of insulin in said one or more saliva samples.

5. The method of claim 4, wherein each of the one or more additional times is within about three hours following ingestion of the foodstuff.

6. The method of claim 1, additionally comprising a step of characterizing the subject's blood or plasma insulin level by comparing the first level of insulin and the second level of insulin in saliva from the subject to a reference data set.

7. The method of claim 6, wherein the reference data set is based upon previously obtained data from one or more individuals having a similar physiological profile to the subject.

8. The method of claim 6, wherein the physiological profile comprises one or more of gender, age, weight, body mass index, waist-to-hip ratio, systolic blood pressure, diastolic blood pressure, metabolism, race, and exercise.

9. The method of claim 6, wherein the reference data set is based upon previously obtained data from the subject.

10. The method of claim 1, wherein if the foodstuff is a high-carbohydrate meal, characterizing the subject's blood or plasma insulin level comprises multiplying the difference between the first level of insulin and the second level of insulin by a factor of between about 2 and about 5.

11. (canceled)

12. The method of claim 10, wherein the high-carbohydrate meal comprises 50% or more by weight carbohydrates.

13. The method of claim 1, wherein if the foodstuff is a low-carbohydrate meal, characterizing the subject's blood plasma insulin level comprises multiplying the difference between the first level of insulin and the second level of insulin by a factor of about 2.

14. The method of claim 13, wherein the low-carbohydrate meal comprises 25% or less by weight carbohydrates.

15. (canceled)

16. A method for determining whether a subject is in a fat-storage mode or a fat-burning mode, said method comprising:
measuring a level of insulin in a saliva sample obtained from a subject; and
comparing the level of insulin to a predetermined value to determine if the subject is in a fat-storage mode or a fat-burning mode,
wherein if the level of insulin is above the predetermined value, the level of insulin is indicative of the fat-storage mode, and
if the level of insulin is below the predetermined value, the level of insulin is indicative of the fat-burning mode.

17. (canceled)

18. The method of claim 16, wherein the predetermined value is a resting state saliva insulin level for the subject, said resting state being at least four hours after their last meal and prior to ingestion of further foodstuff.

19. The method of claim 18, wherein the resting state saliva insulin level for a lean subject is between about 1.0 mU/L and about 2.0 mU/L of salivary insulin.

20. The method of claim 18, wherein the resting state saliva insulin level for an obese subject is between about 2.0 mU/L and about 3.0 mU/L of salivary insulin.

21. The method of claim 16, further comprising, between the steps of measuring and comparing, a step of characterizing the subject's blood or plasma insulin level based upon the level of insulin in saliva, wherein the blood or plasma insulin level is then compared to the predetermined value.

22. The method of claim 21, wherein the predetermined value is a resting state blood or plasma insulin level for the subject, said resting state being at least four hours after their last meal and prior to ingestion of further foodstuff.

23. The method of claim 21, wherein the method is performed after ingestion of a high-carbohydrate or a low-carbohydrate meal.

24.-30. (canceled)
